SEQUENCE LISTING (8 sequences)

SEQ ID NO: 1 - DNA, 20 bp, Artificial Sequence
  TNAP forward primer
agactgcgcctggtagttgt

SEQ ID NO: 2 - DNA, 20 bp, Artificial Sequence
  TNAP reverse primer
cctcctcggaagacactctg

SEQ ID NO: 3 - DNA, 20 bp, Artificial Sequence
  Runx2 forward primer
acgccatagtccctcctttt

SEQ ID NO: 4 - DNA, 20 bp, Artificial Sequence
  Runx2 reverse primer
tcactaccagccaccgagac

SEQ ID NO: 5 - DNA, 23 bp, Artificial Sequence
  GAPDH forward primer
gttgctgtagccaaattcgttgt

SEQ ID NO: 6 - DNA, 23 bp, Artificial Sequence
  GAPDH reverse primer
ggtggtctcctctgacttcaaca

SEQ ID NO: 7 - DNA, 21 bp, Artificial Sequence
  TNAP targeting sequence 1
gcgcaagagacactgaaatat

SEQ ID NO: 8 - DNA, 21 bp, Artificial Sequence
  TNAP targeting sequence 2
actgccatcctgtatggcaat

(19) United States
(12) Patent Application Publication
(10) Pub. No.: US 2021/0301318 A1
(43) Pub. Date: Sep. 30, 2021

(54) A BIOMARKER AND TARGET FOR DIAGNOSIS, PROGNOSIS AND TREATMENT OF ANKYLOSING SPONDYLITIS

(71) Applicants: ACADEMIA SINICA, Taipei City (TW); BUDDHIST TZU CHI MEDICAL FOUNDATION, Hualien City (TW); CHINA MEDICAL UNIVERSITY, Taichung (TW)

(72) Inventors: Kuo-I LIN, Taipei (TW); Shih-Chieh HUNG, Taichung (TW); Chin-Hsiu LIU, New Taipei City (TW)

(73) Assignees: ACADEMIA SINICA, Taipei City (TW); BUDDHIST TZU CHI MEDICAL FOUNDATION, Hualien City (TW); CHINA MEDICAL UNIVERSITY, Taichung (TW)

(21) Appl. No.: 17/258,826

(22) PCT Filed: Jul. 10, 2019
(86) PCT No.: PCT/US2019/041157

§ 371 Date: Jan. 8, 2021

Related U.S. Application Data
(60) Provisional application No. 62/696,020, filed on Jul. 10, 2018.

(51) Int. Cl. C12Q 1/42; G01N 33/50; A61K 45/06; A61K 31/429; A61K 31/663; G01N 33/573; G01N 33/68; C12Q 1/686; C12Q 1/6816

(52) U.S. Cl. CPC C12Q 1/42; G01N 33/5088; G01N 33/5091; A61K 45/06; A61K 31/429; A61K 31/663; G01N 2800/7095; G01N 33/6848; C12Q 1/686; C12Q 1/6816; G01N 2333/916; C12Y 301/03001; G01N 2800/50; G01N 33/573

(57) ABSTRACT

The present invention relates to a biomarker and target for diagnosis, prognosis and treatment of ankylosing spondylitis (AS). The present invention also relates to a method for producing an animal model for AS, an animal model produced therefrom, and a method for screening for an agent pharmaceutically active in the treatment of A S using such animal model.

### RELATED APPLICATIONS

This application claims the benefit of U.S. provisional application No. 62/696,020, filed Jul. 10, 2018 under 35 U.S.C. § 119, the entire content of which is incorporated herein by reference.

### TECHNOLOGY FIELD

The present invention relates to a biomarker and target for diagnosis, prognosis and treatment of ankylosing spondylitis (AS). The present invention also relates to a method for producing an animal model for AS, an animal model produced therefrom, and a method for screening for an agent pharmaceutically active in the treatment of AS using such animal model.

### BACKGROUND OF THE INVENTION

Ankylosing spondylitis (AS) is a type of chronic arthritis characterized by inflammatory spondylitis, peripheral arthritis and enthesitis. Typically, it occurs in young adult males and has a strong association with human leukocyte antigen (HLA)-B27¹. Alongside chronic spinal inflammation, formation of new bone is observed frequently in one-third of patients within 2 years, resulting in ossification and ankylosing of adjacent vertebral bodies (“syndesmophytes”) that can lead to permanent disability². Despite treatments such as non-steroidal anti-inflammatory drugs (NSAIDs), anti-tumor necrosis factor (TNF)-α ameliorating inflammation or interleukin-17 blocker, they are unable to completely arrest syndesmophyte formation³⁻⁹. MRI imaging also provided evidences that the large majority of new syndesmophytes developed in vertebral units without inflammation¹⁰. Hence, syndesmophyte formation could be uncoupled from inflammation¹⁰⁻¹². Further understanding of the pathological mechanism of stromal activation in situ has been the highest priority to prevent spinal ankylosis. Although it has been reported that the osteogenesis of AS patients-derived MSCs is altered^{13,14}, the role of MSCs in the nidus of syndesmophyte and the underlying genetic pathways is largely unknown.

There is a need to develop an approach for diagnosis, prognosis and treatment of AS and a platform to screen for effective agents for treatment of AS.

### SUMMARY OF THE INVENTION

In this invention, we established AS MSCs and found that the enhanced TNAP (an ALPL gene product) in AS MSCs is required for the pathological mechanism of bony apposition induced by AS MSCs, and blockage of TNAP would inhibit abnormal mineralization of AS MSCs. We also established an AS animal model using the AS MSCs which is useful for screening for an agent effective in treating AS. Further, we demonstrated in human subjects that an ALPL gene product (serum TNAP/or bone ALP levels) is highly correlated with ankylosing spondylitis (AS), especially radiographic severity of AS, and thus can serve as a biomarker for diagnosing AS and also for monitoring progression in a patient with AS.

In one aspect, the present invention provides a method for detecting ankylosing spondylitis (AS) and/or predicting the risk of development of radiographic severity of AS, comprising: (i) providing a biological sample from a subject; and (ii) detecting an ALPL gene product as an AS marker in the sample.

In some embodiments, the gene product includes a protein or a RNA transcript.

In some embodiments, the ALPL gene product is a non-specific alkaline phosphatase (TNAP).

In some embodiments, the ALPL gene product is a bone-specific TNAP (BAP).

In some embodiments, the marker is detected with an agent that specifically binds to the ALPL gene product, e.g. an antibody or a primer/probe.

In some embodiments, the detection is performed by an immunoassay, a mass spectrometric assay, a nucleic acid hybridization detection assay, and/or a reverse transferase-polymerase chain reaction (RT-PCR).

In some embodiments, the biological sample is a body fluid sample e.g. blood or serum or a tissue sample e.g. bone marrow slices.

In some embodiments, the method further comprises comparing the results of the detection with a reference level and identifying the subject as having AS and/or at risk of development of radiographic severity of AS, if the comparison shows an elevated level of the ALPL gene product.

In some embodiments, the method further comprises applying a further AS diagnostic assay to the subject to confirm AS occurrence or the risk of development of radiographic severity of AS.

In some embodiments, the radiographic severity includes one or more radiographic features of AS selected from the group consisting of erosion, sclerosis, squaring, syndesmophyte formation, bony bridge, spinal ankylosing and any combination thereof.

In some embodiments, the method further comprises treating the subject with anti-AS therapeutic methods (e.g. surgery and/or physical therapy) and/or medicaments (e.g. corticosteroids, non-steroidal anti-inflammatory drugs (NSAISs), an immunosuppressant, a tumor necrosis factor-alpha (TNF-α) blocker inhibitor, an anti-interleukin-6 inhibitor, an interleukin 17A inhibitor, and/or a TNAP inhibitor (including a BAP inhibitor)).

In particular, the method of the present invention is useful for monitoring progression of AS in an AS patient, comprising

(a) providing a first biological sample from the patient at a first time point;

(b) providing a second biological sample from the patient at a second time point, which is later than the first time point;

(c) measuring the levels of a ALPL gene product as an AS marker in the first and second biological samples; and

(d) determining AS progression in the patient based on the levels of ALPL gene product in the first and second biological samples, wherein an elevated level of ALPL gene product in the second biological sample as compared to that in the first biological sample is indicative of AS progression.

In another aspect, the present invention provides a TNAP inhibitor for use in treatment of AS. Also provided is a method for treating AS, comprising administering a therapeutically effective amount of a TNAP inhibitor to a subject in need. Use of a TNAP inhibitor for manufacturing a medicament for treating AS is also described herein.

In some embodiments, the method is effective in reducing or alleviating one or more conditions of AS, in particular radiographic severity.

In some embodiments, the TNAP inhibitor is selected from the group consisting of levamisole, beryllium sulfate tetrahydrate, pamidronate or any combination thereof.

In some embodiments, the TNAP inhibitor is administered in combination with conventional anti-AS therapeutic methods (e.g. surgery and/or physical therapy) and/or other medicaments (e.g. corticosteroids, non-steroidal anti-inflammatory drugs (NSAISs), an immunosuppressant, a tumor necrosis factor-alpha (TNF-α) blocker inhibitor, an anti-interleukin-6 inhibitor, and/or an interleukin 17A inhibitor).

In some embodiments, the method of the invention does not alter overall bone mineral density (BMD).

In a further aspect, the present invention provides a method for producing an animal model for AS, comprising the steps of:

(i) providing marrow mesenchymal stem cells (BMSCs) from bone marrow in the spine afflicted with spinal ankyloses from a AS patient;

(ii) implanting the BMSCs in areas adjacent of lamina of lumbar spine segments (e.g. L4 to L5) in an immunodeficient mice; and

(iii) growing the mice in a condition suitable to induce one or more symptoms/conditions of AS, in particular to develop radiographic severity (e.g. erosion, sclerosis, squaring, syndesmophyte formation, bony bridge, spinal ankylosing and the like).

Also provided is an animal model for AS prepared by the method as described herein.

In some embodiments, the animal model is a mammal such as a mouse, a rat, a rabbit, a pig, a cow, a dog, and a monkey.

Further provided is a method for screening for an agent effective in treating AS, comprising the steps of:

(i) administering a test agent to an animal model as described herein; and

(ii) determining whether at least one of AS condition/symptom is reduced or alleviated.

A test agent useful in reducing or alleviating at least one of AS condition/symptom in the animal model is deemed a candidate medicament for treating AS.

Also provided is a kit for performing the method as described herein, comprising an agent (e.g. an antibody) that specifically binds to the AS marker, and instructions for performing the method.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A-1I shows Runx2-independent accelerated mineralization in AS MSCs under osteogenic induction. FIG. 1A, ARS staining of enhanced mineralization in AS MSCs cultured under osteogenic condition at indicated days as compared with N MSCs. FIG. 1B, Representative quantification result of ARS staining by optical density (O.D.) measurement showing the rate in mineralization between three AS MSCs and three N MSCs at indicated days. FIG. 1C, RT-QPCR of Runx2 mRNA levels in AS MSCs and N MSCs at days 0 and 7 under osteogenic induction. FIGS. 1D-1F, MSCs were transfected with shRNA against Runx2 (shRunx2) or control (shCtrl) lentiviral vector, and then cultured under osteogenic condition. FIG. 1D, RT-QPCR showing the knockdown efficiency by two independent shRunx2. FIG. 1E, ARS staining showing effects of Runx2 knockdown on the mineralization of AS MSCs. FIG. 1F, Quantification result of (FIG. 1E) assayed by O.D. measurement of ARS staining. FIG. 1G, Immunofluorescence staining of AS MSCs and N MSCs at day 14 under osteogenic induction with DAPI (blue) and osteoadherin-specific antibody (green). FIG. 1H and FIG. 1I, RT-QPCR showing the mRNA levels of osteocalcin (FIG. 1H) and collagen 1A1 (FIG. 1I) in AS MSCs and N MSCs at day 7 under osteogenic induction. Results are from at least three independent experiments. Data represent the mean±standard error of mean (SEM) (n=3). * P<0.05, ** P<0.01, *** P<0.001, **** P<0.0001 by Student's t-test. Scale bar=200 μm (a, e); 20 μm (g).

FIGS. 2A-2M shows enhanced expression of TNAP is essential for abnormal mineralization in AS MSCs under osteogenic induction. FIGS. 2A-2C, ALP activity and TNAP levels in AS MSCs and N MSCs at 0 and 7 days after osteogenic induction as determined by ALP enzyme activity (FIG. 2A), TNAP mRNA levels measured by RT-QPCR (FIG. 2B), and immunoblot (FIG. 2C). FIGS. 2D-2E, The effects of TNAP inhibitors (100 μM levamisole, 100 μM beryllium sulfate, or 1 μg/ml pamidronate) on the mineralization in AS MSCs under osteogenic induction as determined by ARS staining (FIG. 2D) with quantification (FIG. 2E). FIGS. 2F-2H, ALP activity (FIG. 2F), TNAP mRNA (FIG. 2G) and protein levels (FIG. 2H) were suppressed by shTNAP in AS MSCs after 7 days of osteogenic induction. FIGS. 2I-2J, Inhibition of accelerated mineralization in AS MSCs by two shTNAP under osteogenic induction as determined by ARS staining (FIG. 2I) with quantification (FIG. 2J). FIGS. 2K-2M, Overexpression of TNAP via lentiviral transduction (p-TNAP) in N MSCs as determined by ARS staining (FIG. 2K) with quantification (FIG. 2L). FIG. 2M, Immunoblot shows the expression of TNAP protein. Results are from at least three independent experiments. Data represent the mean±SEM. * P<0.05, *** P<0.001, **** P<0.0001 by Student's t-test. Scale bar=200 μm (FIG. 2D, FIG. 2I, FIG. 2K)

FIGS. 3A-3D shows that TNAP blockade inhibits new bony apposition induced by AS MSCs in NOD-SCID mice. FIGS. 3A-3D, Representative images of lumbar spine micro-computed tomography of NOD-SCID mice implanted with AS MSCs or N MSCs adjacent to the right lamina of lumbar spine segment L4-5 (FIG. 3A), with AS MSCs transduced with shCtrl or shTNAP (FIG. 3B), or with AS MSCs plus daily oral administration with H₂O, levamisole (10 mg/kg), beryllium sulfate (7.5 mg/kg), or pamidronate (0.3 mg/kg) (FIG. 3C). Images were taken 3 weeks after implantation. Longitudinal view over L4-L6 (left), longitudinal view at high magnification over L4 (middle) and cross-sectional view over L4 (right) are shown. New bony appositions are highlighted by a red rectangle (middle) and white arrow (right). Representative images of three mice in each group are shown. FIG. 3D, The quantitative volumes of new bony apposition (mm³) in each group. Data are the mean SEM (n=9 in each group of AS MSC or N MSCs, n=9 in each group of AS MSCs transduced with shTNAP or shCtrl, n=9 in each group of AS MSCs plus daily oral administration of H₂O, levamisole, beryllium sulfate or pamidronate). * P<0.05, ** P<0.01, *** P<0.001 by Student's t-test.

FIGS. 4A-4D shows that TNAP in the BM and serum from AS patients are elevated significantly as compared with those from healthy individuals, and is associated with radiographic severity in AS patients. FIG. 4A, IHC staining of the BM from AS patients and normal controls with a TNAP-specific antibody. Inset represents high magnification of the boxed area. FIGS. 4B-4D, Double IHC staining with TNAP antibody (in brown) and indicated second primary antibodies (in blue). The co-localization of TNAP/CD68 (monocyte lineage) (FIG. 4B), TNAP/myeloperoxidase (MPO) (myeloid lineage) (FIG. 4C), TNAP/CD44 (MSC) (FIG. 4D) were visualized as dark purple color. Inset represents high magnification (400×) from the boxed area; upper image is the microscopic image and lower image is the composite pseudo-colored image by spectral unmixing technique with the spectral library (staining with TNAP antibody in brown and staining with secondary antibody in blue). The co-localization of TNAP/MPO, TNAP/CD68, and TNAP/CD44 was presented as turquoise color. Scale bar=50 μm (FIGS. 4A-4D).

FIG. 5 shows that enhanced expression of TNAP is required for abnormal mineralization in AS MSCs under osteogenic induction. MSCs were cultured in growth medium (GM) with or without addition of P-glycerophosphate (BGP), and mineralization was determined by ARS staining. (left panel) ARS staining of mineralization in AS and N MSCs (scale bar=200 μm). (right panel) Quantification result of ARS staining in (t). Data are the mean±SEM. Results were from two independent experiments undertaken in triplicate. * P<0.05, **P<0.01, ***P<0.001, ****P<0.0001 by Student's t test.

FIG. 6 shows the bone mineral density 12 weeks after oral administration of TNAP inhibitors. Femoral bone mineral density was measured by micro-CT in NOD-SCID mice following implantation with AS MSCs and daily oral administration of H₂O (n=4), levamisole (10 mg/kg) (n=3), beryllium sulfate (7.5 mg/kg) (n=3), or pamidronate (0.3 mg/kg) (n=3).

FIG. 7 shows the TNAP expression in the BM of non-AS patient control. IHC staining of the BM from non-AS patient with a TNAP-specific antibody. Scale bar=50 prm. Inset represents high magnification of the boxed area.

FIG. 8 shows the expression of cytokines secreted by AS MSCs and N MSCs under osteogenic induction at days 0-3, 3-7, and 7-10. Data are the mean±SEM. Results were from two independent experiments undertaken in triplicate.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of 10% around the cited value.

As used herein, the term “nucleic acid fragment,” “nucleic acid” and “polynucleotide,” used interchangeably herein, refer to a polymer composed of nucleotide units, including naturally occurring nucleic acids, such as deoxyribonucleic acid (“DNA”) and ribonucleic acid (“RNA”) as well as nucleic acid analogs including those which have non-naturally occurring nucleotides. Thus, these terms include, but are not limited to, single-, double-, or multi-stranded DNA or RNA, genomic DNA, cDNA, mRNA, DNA-RNA hybrids, or a polymer comprising purine and pyrimidine bases or other natural, chemically or biochemically modified, non-natural, or derivatized nucleotide bases. It will be understood that when a nucleic acid fragment is represented by a DNA sequence (i.e., A, T, G, C), this also includes an RNA sequence (i.e., A, U, G, C) in which “U” replaces “T.”

As used herein, the term “primer” as used herein refers to a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase. For example, primers for ALPL gene, as used herein, respectively, are those which are capable to hybridize to the nucleotide sequence of the individual target genes to initiate nucleotide polymerization and produce the nucleotide products as expected based on the design of the sequences of the primers. Examples of the primers for ALPL gene are

(SEQ ID NO: 1)
AGACTGCGCCTGGTAGTTGT
and
(SEQ ID NO: 2)
CCTCCTCGGAAGACACTCTG.

As used herein, the term “probe” as used herein refers to a defined nucleic acid segment (or nucleotide analog segment, e.g., polynucleotide as defined herein) which can be used to identify a specific polynucleotide sequence present in samples during hybridization, said nucleic acid segment comprising a nucleotide sequence complementary of the specific polynucleotide sequence to be identified. Typically, a probe can produce a detectable signal since it is labeled in some way, for example, by incorporation of a reporter molecule such as a fluorophore or radionuclide or an enzyme. For example, probes for ALPL gene, as used herein, respectively, are those which are capable to specifically hybridize to the corresponding nucleotide sequence of the individual target genes and produce detectable signals caused by such hybridization.

As used herein, the term “hybridization” as used herein shall include any process by which a strand of nucleic acid joins with a complementary strand through base pairing. Relevant technologies are well known in the art and described in, for example, Sambrook et al., Molecular Cloning: A Laboratory Manual, 2^{nd} ed., Cold Spring Harbor Laboratory Press (1989), and Frederick M. A. et al., Current Protocols in Molecular Biology, John Wiley & Sons, Inc. (2001). Typically, stringent conditions are selected to be about 5 to 30° C. lower than the thermal melting point (Tₘ) for the specified sequence at a defined ionic strength and pH. More typically, stringent conditions are selected to be about 5 to 15° C. lower than the Tₘ for the specified sequence at a defined ionic strength and pH. For example, stringent hybridization conditions will be those in which the salt concentration is less than about 1.0 M sodium (or other salts) ion, typically about 0.01 to about 1 M sodium ion concentration at about pH 7.0 to about pH 8.3 and the temperature is at least about 25° C. for short probes (e.g., 10 to 50 nucleotides) and at least about 55° C. for long probes (e.g., greater than 50 nucleotides). An exemplary non-stringent or low stringency condition for a long probe (e.g., greater than 50 nucleotides) would comprise a buffer of 20 mM Tris, pH 8.5, 50 mM KCl, and 2 mM MgCl₂, and a reaction temperature of 25° C.

As used herein, the term “encode” as used herein refers to the inherent property of specific sequences of nucleotides in a polynucleotide (e.g., a gene, a cDNA, or an mRNA) to serve as templates for synthesis of a gene product having either a defined sequence of nucleotides (i.e., rRNA, tRNA and mRNA) or a defined sequence of amino acids and the biological properties resulting therefrom.

As used herein, the term “expression” as used herein refers to the realization of genetic information encoded in a gene to produce a gene product such as an unspliced RNA, an mRNA, a splice variant mRNA, a polypeptide or protein, a post-translationaly modified polypeptide, a splice variant polypeptide and so on.

As used herein, the term “expression level” refers to the amount of a gene product expressed by a particular gene in cells which can be determined by any suitable method known in the art.

As used herein, the terms “polypeptide” and “protein,” used interchangeably herein, refer to a polymeric form of amino acids of any length, which can include coded and non-coded amino acids, chemically or biochemically modified or derivatized amino acids, and polypeptides having modified peptide backbones.

As used herein, the term “antibody” means an immunoglobulin protein which is capable of binding an antigen. Antibody as used herein is meant to include the entire antibody as well as any antibody fragments (e.g., F(ab′).sub.2, Fab′, Fab, Fv) capable of binding the epitope, antigen, or antigenic fragment of interest. Antibodies of the invention are immunoreactive or immunospecific for and therefore specifically and selectively bind to a protein of interest, e.g., such as an ALPL gene product e.g. a non-specific alkaline phosphatase (TNAP). Antibodies for the proteins of interest are preferably immunospecific, i.e., not substantially cross-reactive with related materials, although they may recognize their homologs across species. The term “antibody” encompasses all types of antibodies (e.g., monoclonal and polyclonal).

According to the present invention, an ALPL gene product can be used as an AS marker for detecting occurrence of ankylosing spondylitis (AS) and/or predicting the risk of development of radiographic severity of AS.

As used herein, a biological marker (or called biomarker or marker) is a characteristic that is objectively measured and evaluated as an indicator of normal or abnormal biologic processes/conditions, diseases, pathogenic processes, or responses to treatment or therapeutic interventions. Markers can include presence or absence of characteristics or patterns or collections of the characteristics which are indicative of particular biological processes/conditions. A marker is normally used for diagnostic and prognostic purposes. However, it may be used for therapeutic, monitoring, drug screening and other purposes described herein, including evaluation the effectiveness of a AS therapeutic.

“Diagnosis” as used herein generally includes determination as to whether a subject is likely affected by a given disease, disorder or dysfunction. The skilled artisan often makes a diagnosis on the basis of one or more diagnostic indicators, i.e., a marker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. It would be understandable that a positive prognosis typically refers to a beneficial clinical outcome or outlook, such as less symptoms of AS, whereas a negative prognosis typically refers to a negative clinical outcome or outlook, such as more symptoms of AS. In certain embodiments, the symptoms of AS are radiographic severity including one or more radiographic features selected from the group consisting of erosion, sclerosis, squaring, syndesmophyte formation, bony bridge, spinal ankylosing and any combination thereof.

As used herein, the terms “subject,” “individual” and “patient” can refer to a mammalian subject for whom diagnosis, prognosis, treatment, or therapy is needed, particularly humans. Other subjects may include cattle, dogs, cats, guinea pigs, rabbits, rats, mice, horses, and so on.

As used herein, an “aberrant level” can refer to a level that is increased compared with a reference level. For example, an aberrant level can be higher than a reference level by more than 10%, 20%, 30%, 40%, 50%, 60%, 70%, 80%, 90% or 100%. In some embodiments, the expression level of a biomarker as described herein in a subject to be tested is compared to a standard level based on historical values. For example, the standard level can be set based on an average or median expression level of such biomarker in corresponding biological samples obtained from a cohort of subjects. For instance, the cohort of subjects can be a group of AS patients enrolled in a clinical trial. In some embodiments, a reference level can refer to the level measured in normal individuals or samples such as tissues or cells that are not diseased.

As used herein, “low expression” and “high expression” for a biomarker as used herein are relative terms that refer to the level of the biomarker found in a sample. In some embodiments, low and high expression can then be assigned to each sample based on whether the expression of such biomarker in a sample is above (high) or below (low) the average or median expression level. In some embodiments, low and high expression can be determined by comparison of the biomarker expression level in a non-diseased sample, where low expression can refer to a lower or comparable expression level to the expression level in a non-diseased sample, and high expression can refer to a higher expression level to the expression level in a non-diseased.

As used herein, the term “ALPL” is a gene encoding an alkaline phosphatase (ALP), tissue-nonspecific isozyme (i.e. a non-specific alkaline phosphatase (TNAP)) in human. ALP is a large superfamily of unbiquitous ectoenzyme that can catalyze dephosphorylation and transphosphrylation reaction. It consists of four isoenzymes, including TNAP, placental, germ cell and intestinal ALP encoded by separate genes. The last three are located together on chromosome 2, while the first one, tissue non-specific form is located on chromosome 1. The product of this gene is a membrane bound glycosylated enzyme that is not expressed in a particular tissue and is, thus, referred to as the tissue-nonspecific form of the enzyme. TNAP encoded by ALPL gene is distributed in liver/bone/kidney tissues with alternative splicing transcript variants^{20,21}. It hydrolyzes pyrophosphate and provides inorganic phosphate to promote mineralization^{20,21}. The nucleotide sequences of the biomarker genes as described above and the corresponding amino acid sequences of their gene products are well known in the art. For example, Homo sapiens ALPL, transcript variant 1, mRNA/protein: NM_000478/NP_000469.3; Homo sapiens ALPL, transcript variant 2, mRNA/protein: NM_001127501.4/NP_001120973.2; Homo sapiens ALPL, transcript variant 3, mRNA/protein: NM_001177520.3/NP_001170991.1; Homo sapiens ALPL, transcript variant 4, mRNA/protein: NM_001369803.2/NP_001356732.1; Homo sapiens ALPL, transcript variant 5, mRNA/protein::NM_001369804.2/NP_001356733.1.

To perform the methods described herein, a biological sample can be obtained from a subject in need and the marker in the biological sample can be detected or measured via any methods known in the art, such as an immunoassay, a mass spectrometric assay, a nucleic acid hybridization detection assay, and/or a reverse transferase-polymerase chain reaction (RT-PCR). A biological sample can be a body fluid sample e.g. blood or serum or a tissue sample e.g. bone marrow slices. The detection of the marker(s) may be quantitative or qualitative. In one embodiment, a sample obtained from a subject in need is analyzed for the presence or absence of the marker(s). If the marker(s) is detected in a sample obtained from a subject in need, the subject is identified as having AS or at the risk of development of radiographic severity of AS. The reference level can represent the level of the marker(s) in a control sample, which may be obtained from a normal subject or a pool of such subjects. In some examples, the level of the marker(s) in a control sample is undetectable in a control sample (i.e. the reference value being 0) using a routine assay e.g. immunoassays, and the presence of the marker as detected in a biological sample from a subject using the same assay can indicate AS occurrence or the risk of development of radiographic severity of AS. In some examples, the level of the marker(s) can be measured at different time points in order to monitor the progression of AS. For example, two biological samples are obtained from a candidate subject at two different time points. If a trend of increase in the level of the marker(s) is observed over time, for example, the level of the marker(s) in a later obtained sample is higher than that in an earlier obtained sample, the subject is deemed as having a negative prognosis of AS, in particular an increased level of radiographic severity of AS.

The presence and amount of the biomarker as described herein in a biological sample can be determined by routine technology.

In some embodiments, the presence and/or amount of the biomarker as described herein can be determined by mass spectrometry, which allows direct measurements of the analytes with high sensitivity and reproducibility. A number of mass spectrometric methods are available. Examples of mass spectrometry include, but are not limited to, liquid chromatography-mass spectrometry (LC-MS), liquid chromatography tandem mass spectrometry (LC-MS-MS), electrospray ionization mass spectrometry (ESI-MS), matrix-assisted laser desorption ionization/time of flight (MALDI-TOF), and surface-enhanced laser desorption ionisation/time of flight (SELDI-TOF). One certain example of this approach is tandem mass spectrometry (MS/MS), which involves multiple steps of mass selection or analysis, usually separated by some form of fragmentation.

In some embodiments, the presence and/or amount of a biomarker can be determined by an immunoassay. Examples of the immunoassays include, but are not limited to, Western blot, enzyme-linked immunosorbent assay (ELISA), radioimmunoassay (RIA), radioimmunoprecipitation assay (RIPA), immunofluorescence assay (IFA), ELFA (enzyme-linked fluorescent immunoassay), electrochemiluminescence (ECL), and Capillary gel electrophoresis (CGE). In some examples, the presence and/or level of a biomarker can be determined using an agent specifically recognizes said biomarker, such as an antibody that specifically binds to the biomarker.

In other embodiments, the presence and/or amount of a biomarker can be determined by measuring mRNA levels of the one or more genes. Assays based on the use of primers or probes that specifically recognize the nucleotide sequences of the genes as described may be used for the measurement, which include but are not limited to reverse transferase-polymerase chain reaction (RT-PCR) and in situ hybridization (ISH), the procedures of which are well known in the art. Primers or probes can readily be designed and synthesized by one of skill in the art based on the nucleic acid region of interest. It will be appreciated that suitable primers or probes to be used in the invention can be designed using any suitable method in view of the nucleotide sequences of the genes of interest as disclosed in the art.

Antibodies as used herein may be polyclonal or monoclonal. Polyclonal antibodies directed against a particular protein are prepared by injection of a suitable laboratory animal with an effective amount of the peptide or antigenic component, collecting serum from the animal, and isolating specific sera by any of the known immunoadsorbent techniques. Animals which can readily be used for producing polyclonal antibodies as used in the invention include chickens, mice, rabbits, rats, goats, horses and the like.

In particular embodiments, an ALPL gene product as an AS biomarker is a TNAP protein, including a bone-specific TNAP (BAP), a liver-specific TNAP, and/or a kidney-specific TNAP. In some embodiments, the TNAP protein as an AS biomarker includes a bone-specific TNAP (BAP) and/or a liver-specific TNAP. In one certain example, the TNAP protein as an AS biomarker is a bone-specific TNAP (BAP).

In some embodiments, the amount of a biomarker in the sample derived from the candidate individual can be compared to a standard value to determine whether the candidate individual has a negative prognosis of AS. The standard value may represent the average or median amount of a biomarker as described herein in a population of AS. Typically, such population of AS patients are chosen to be matched to the candidate individual in, for example, age and/or ethnic background. Preferably, such population of AS patients and the candidate individual are of the same species.

When an individual, such as a human patient, is diagnosed as having a negative prognosis, the individual may undergo further testing (e.g., routine physical testing, including surgical biopsy or imaging methods, such as X-ray imaging, magnetic resonance imaging (MRI), or ultrasound) to confirm the occurrence of the disease and/or to determine the stage and progression of the disease.

In some embodiments, the methods described herein can further comprise treating the AS patient to at least relieve symptoms associated with the disease. The treatment can be any conventional anti-AS therapy (e.g. surgery and/or physical therapy) and/or medicaments (e.g. corticosteroids, non-steroidal anti-inflammatory drugs (NSAISs), an immunosuppressant, a tumor necrosis factor-alpha (TNF-α) blocker inhibitor, an anti-interleukin-6 inhibitor, an interleukin 17A inhibitor, and/or a TNAP inhibitor as described herein).

Also provided is a kit for performing the method of the invention. Specifically, the kit comprises a reagent (e.g., an antibody, a primer, a probe, or a labeling reagent) that can specifically detect the marker(s) as described herein. The kit can further instructions for using the kit to detect the presence or amount of the marker(s) in a biological sample for predicting prognosis and/or monitoring progression of the disease. The components including the detection reagents as described herein can be packaged together in the form of a kit. For example, the detection reagents can be packaged in separate containers, e.g., a nucleic acid (a primer or a probe) or antibody (either bound to a solid matrix or packaged separately with reagents for binding them to the matrix), a control reagent (positive and/or negative), and/or a detectable label, and the instructions (e.g., written, tape, VCR, CD-ROM, etc.) for performing the assay can also be included in the kit. The assay format of the kit can be a Northern hybridization, a chip or an ELISA, for example. Further provided is use of such reagent for performing a method for predicting prognosis and/or monitoring progression of the disease. The reagent may be mixed with a carrier e.g. a pharmaceutically acceptable carrier to form a composition for the detection or diagnosis purpose. Examples of such carrier include injectable saline, injectable distilled water, an injectable buffer solution and the like.

In another aspect, the present invention is based on the unexpected findings that a TNAP inhibitor may be used as an active ingredient for treating AS. Accordingly, the present invention relates to treatment of AS by means of inhibition of TNAP.

Specifically, inhibition of TNAP (expression or activity) is performed by a TNAP inhibitor. Examples of a TNAP inhibitor can include nucleic acid molecules (e.g. an anti-sense nucleic acid molecule directed to a corresponding gene or a small interfering RNA (siRNA) directed toward a corresponding nucleic acid), polypeptides (e.g. antibodies), or a small molecule TNAP inhibitory compound.

As used herein, the term “small molecule” refers to organic or inorganic molecules either synthesized or found in nature, generally having a molecular weight less than 10,000 grams per mole, particularly less than 5,000 grams per mole, particularly less than 2,000 grams per mole, and particularly less than 1,000 grams per mole. In some embodiments, a small molecule as described herein refers to a non-polymeric, e.g. non-protein or nucleic acid based, chemical molecule. Examples of small molecules as a TNAP inhibitor include but are not limited to levamisole, beryllium sulfate tetrahydrate, pamidronate.

| TNAP inhibitors | Structure/chemical name |
|---|---|
| levamisole | 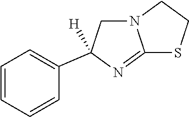 [SMILES: c1ccc([C@H]2CN3CCSC3=N2)cc1] |
| | (6S)-6-phenyl-2H,3H,5H,6H-imidazo |
| | [2,1-b][1,3]thiazole |
| beryllium sulfate | BeSO4•4H₂O |
| tetrahydrate | |
| pamidronate | 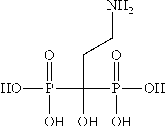 [SMILES: NCCC(O)(P(=O)(O)O)P(=O)(O)O] |
| | 3-amino-1-hydroxypropane-1,1- |
| | diyl)bis(phosphonic acid) |

The term “treating” or “treatment” as used herein refers to the application or administration of a composition including one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression of the disorder or the symptom or condition thereof.

The term “effective amount” used herein refers to the amount of an active ingredient to confer a desired therapeutic effect in a treated subject. For example, an effective amount for treating AS can be an amount that can prohibit, improve, alleviate, reduce or prevent one or more symptoms or conditions or progression thereof, in particular radiographic features of AS e.g. erosion, sclerosis, squaring, syndesmophyte formation, bony bridge, and spinal ankylosing. The symptoms may be determined and evaluated using methods known in the art e.g. X-ray or MRI image. Persons skilled in the art may determine the dosage in each case based on the disclosure herein, established methods, and their own experience.

In some embodiments, an effective amount of the active ingredient may be formulated with a pharmaceutically acceptable carrier into a pharmaceutical composition of an appropriate form for the purpose of delivery and absorption. Depending on the mode of administration, the pharmaceutical composition of the present invention preferably comprises about 0.1% by weight to about 100% by weight of the active ingredient, wherein the percentage by weight is calculated based on the weight of the whole composition.

As used herein, “pharmaceutically acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder.

The composition of the present invention may be delivered via any physiologically acceptable route, such as oral, parenteral (such as intramuscular, intravenous, subcutaneous, and intraperitoneal), transdermal, suppository, and intranasal methods. Regarding parenteral administration, it is preferably used in the form of a sterile water solution, which may comprise other substances, such as salts or glucose sufficient to make the solution isotonic to blood. The water solution may be appropriately buffered (preferably with a pH value of 3 to 9) as needed. Preparation of an appropriate parenteral composition under sterile conditions may be accomplished with standard pharmacological techniques well known to persons skilled in the art, and no extra creative labor is required.

According to the present invention, a TNAP inhibitor can be administered to a subject in need for treating AS. Therefore, the present invention provides a method for treating AS by administering a TNAP inhibitor as described herein or a composition comprising the same to a subject in need. In particular, the TNAP inhibitor as described herein is administered in an amount effective in (i) inhibiting the enzymatic activity or expression level of TNAP, and (ii) reducing or preventing one or more symptoms or conditions or progression thereof, in particular radiographic features of AS e.g. erosion, sclerosis, squaring, syndesmophyte formation, bony bridge, and spinal ankylosing. In some embodiments, the TNAP inhibitor as described herein is administered in an amount not substantially reduce bone density (e.g. overall bone mineral density (BMD)) in the AS patient. More particularly, the TNAP inhibitor as described herein is administered in an amount not resulting in osteoporosis in the AS patient.

In some embodiments, a TNAP inhibitor as described herein can be administered in combination with conventional anti-AS therapeutic methods e.g. surgery and/or physical therapy, and/or known medicament for AS, including but are not limited to corticosteroids, non-steroidal anti-inflammatory drugs (NSAISs), an immunosuppressant, a tumor necrosis factor-alpha (TNF-α) blocker inhibitor, an anti-interleukin-6 inhibitor, an interleukin 17A inhibitor.

In a further aspect, the present invention provides a method for producing a non-human animal model for AS. Also provided is a non-human animal model for AS thus prepared.

In some embodiments, the method comprises the steps of
(i) providing marrow mesenchymal stem cells (BMSCs) from bone marrow in the spine afflicted with spinal ankyloses from a AS patient;
(ii) implanting the BMSCs in areas adjacent of lamina of lumbar spine segments in an immunodeficient non-human animal; and
(iii) growing the animal in a condition suitable to induce one or more symptoms/conditions of AS.

In certain embodiments, the lumbar spine segments is L4 to L5.

In certain embodiments, the AS symptoms/conditions include radiographic severity (e.g. erosion, sclerosis, squaring, syndesmophyte formation, bony bridge, spinal ankylosing and any combination thereof).

In some embodiments, the animal model is a mammal such as a mouse, a rat, a rabbit, a pig, a cow, a dog, and a monkey.

According to the present invention, the animal model can be used for screening for a candidate agent for treating AS.

Therefore, the present invention further provides a method as a platform for screening for a candidate anti-AS agent based on the animal model as described herein.

In particular, the method comprises the steps of (i) administering a test agent to the animal model, and (ii) measuring whether at least one of the AS symptoms/conditions is reduced or alleviated, wherein reduction or alleviation of at least one of the AS symptoms/conditions in the animal model via administration of the test agent indicates that the test agent is a candidate agent useful for treating AS.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

1. Material and Methods

1.1 Human Subjects

All donors provided written informed consent before sampling in accordance with the Declaration of Helsinki. The study protocol was approved by the Research Ethics Committees of Taipei Tzu Chi Hospital, Buddhist Tzu Chi Medical Foundation, Taipei Veteran General Hospital, Academic Sinica (all of which are in Taiwan) and Royal National Hospital for Rheumatic Diseases, UK.

Bone marrow (BM) samples were obtained for MSCs isolation from ankylosing spondylitis (AS) patients who had undergone spinal osteotomy (A1, A2, A3) and non-AS controls (C1, C2, C3). All BM donors for MSCs isolation are Taiwanese. In addition, two independent cohorts were recruited for the study of predictive peripheral blood marker for high risk AS patients with propensity for radiographic progression. The “Taiwanese cohort” comprised of 104 patients who fulfilled the modified New York Criteria for AS diagnosis and 50 healthy controls in Taipei Tzu Chi Hospital. A part of this population (37 AS patients who had retrospective follow-up of radiographic change) consisted the longitudinal AS subgroup of “Taiwanese cohort”. The “British cohort” of 184 AS patients were recruited at Royal National Hospital for Rheumatic Diseases, UK. Clinical assessments and radiographic assessments were obtained on the same day. Clinical assessments included the followings: AS disease duration; serologic tests (HLA-B27, C-reactive protein or erythrocyte sedimentation rate); disease activity scoring with a validated version of the Bath Ankylosing Spondylitis Disease Activity Index (BASDAI; range, 0-10; scores of >4 suggest suboptimal control of disease). Two reliable scoring systems, a validated version of the Bath Ankylosing Spondylitis Radiology Index-total score (BASRI-total) was obtained in Taiwanese cohort and modified Stoke Ankylosing Spondylitis Spinal Score (mSASSS) was employed in both cohorts. The BASRI-total score was a combined score for the lumbar spine, cervical spine, sacroiliac joints and hips (range: 2-20). In the mSASSS, the anterior parts of the cervical and lumbar vertebrae were assessed by scoring at 24 spine levels (range 0-3 at each spine level, range: 0-72).

The Bath Ankylosing Spondylitis Functional Index (BASFI) was also recorded. It is a validated index for determining the degree of functional limitation, with the mean visual analogue scale (0 being “easy” and 10 “impossible) being used to answer the questions in the test. The mean of the ten scales gives the BASFI score a value between 0 and 10. Besides, the Bath Ankylosing Spondylitis Global Score (BAS-G), which reflects the effect of AS on the patient's well-being, was obtained. The mean of the two scores gives a BAS-G score of 0-10. The higher the score, the greater the perceived effect of the disease on the patient's well-being.

1.2 Isolation of BM-MSCs within Tissues Affected by Spinal Ankylosis from AS Patients

MSCs from the BM within tissues involved in spinal ankylosis from three AS patients (A1, A2, A3) that had undergone spinal wedge osteotomy (AS MSCs) were isolated. MSCs of three non-AS individuals (C1, C2, C3) who underwent traumatic surgery from BM at similar sites (N MSCs) were used as controls. All are Taiwanese. These specimens were minced finely and digested with 1 mg/mL collagenase D (Roche, Basel, Switzerland) after rinsing with alpha-minimum essential medium (u-MEM; Gibco, Grand Island, N.Y., USA) containing antibiotic-antimycotic solution. After overnight incubation at 37° C., digested tissues were filtered through a 40-μm nylon filter to remove debris. Cells were collected by centrifugation and plated in culture dishes to allow attachment. Non-adherent cells were removed by changing the medium within 48 h and then every 3 days. Upon reaching confluence, cells were harvested and sub-cultured. At passage 3, aliquots of primary MSCs were cryopreserved in liquid nitrogen and expanded for experimentation. The same passages (p3 to p5) of AS MSCs and N MSCs were compared.

1.3 Characterization of MSCs

To analyze cell-surface expression of the markers of MSCs, MSCs were labeled with the following anti-human antibodies: CD31-PE, CD34-PE, CD44-PE, CD29-FITC, CD45-FITC, and CD105-FITC (all from Ancell, Bayport, Minn., USA). Mouse isotype antibodies (Ancell) served as controls. Overall, 100,000 labeled cells were acquired and analyzed using a FACScanto Flow Cytometer (BD Biosciences, Franklin Lakes, N.J., USA). FCS Express v3.0 (De Novo Software, Glendale, Calif., USA) was used for data analyses.

To characterize the tri-lineage differentiation potential of MSCs, MSCs were treated in three culture conditions. The first culture condition was osteogenic induction, whereby α-MEM was supplemented with 10% fetal bovine serum (Gibco), 50 μg/mL ascorbate-2 phosphate (Sigma-Aldrich, St. Louis, Mo., USA), 10 nmol/L dexamethasone (Sigma-Aldrich), and 10 mmol/L β-glycerophosphate (Sigma-Aldrich). The second culture condition was adipogenic induction, whereby α-MEM was supplemented with 10% fetal bovine serum, 50 μg/mL ascorbate-2 phosphate, 100 nmol/L dexamethasone, 50 μg/mL indomethacin (Sigma-Aldrich) and 10 μg/mL insulin (Gibco). The third culture condition was chondrogenic induction, whereby serum-free α-MEM was supplemented with 50 μg/mL ascorbate-2 phosphate, 100 nmol/L dexamethasone, 50 mg/mL insulin-transferrin-selenium-Premix (Gibco), and 10 ng/mL transforming growth factor-01 (Prepotech, Rocky Hill, N.J., USA). The medium was changed every 3 days. After appearance of the morphologic features of differentiation, cells were fixed with 4% paraformaldehyde. Cells treated by osteogenic culture were stained for Alizarin red S (Sigma-Aldrich) for mineralization. Quantification of Alizarin red S staining was conducted by measurement of the optical density (OD) of extracted dye at 550 nm with a plate reader (Molecular Devices, Sunnyvale, Calif., USA). Cells treated in adipogenic or chondrogenic culture conditions were stained with Oil red-O (Sigma-Aldrich) or Alcian Blue (ScyTek, Cache, Utah, USA) to assess adipogenic and chondrogenic differentiation, respectively.

1.4 TNAP Inhibitor Treatment of MSCs Under Osteogenic Induction

The MSCs were seeded at 8×10⁴ cells per 6 well-plate and grown in osteogenic induction medium at 37° C. with 5% C02 incubation, with/without levamisole (100 μM; Sigma-Aldrich) (non-competitive TNAP inhibitor), beryllium sulfate tetrahydrate (competitive TNAP inhibitor) (100 μM; Santa Cruz Biotechnology), or pamidronate (non-competitive TNAP inhibitor) (1 μg/ml; Sigma-Aldrich). After morphological differentiation, cells were subjected to Alizarin red S staining.

1.5 Reverse Transcription Quantitative Polymerase Chain Reaction (RT-QPCR)

Total RNA was extracted using an RNeasy Mini kit (Qiagen, Hilden, Germany) according to manufacturer's instructions. First-strand cDNA was synthesized from 2 μg of total RNA using a High-capacity cDNA Reverse Transcription kit (Applied Biosystems, Waltham, Mass., USA). RT-QPCR was carried out with a SYBR Green PCR Master Mix on a Prism 7300 Sequence Detection system (Applied Biosystems). The relative quantitation of marker genes was done according to the ΔCt method using glyceraldehyde 3-phosphate dehydrogenase (GAPDH) as the internal control gene. The specific primers used for RT-QPCR are shown below.

| | Sequence of | Sequence of |
|---|---|---|
| Genes | forwards primer | reverse primer |
| TNAP | AGACTGCGCC | CCTCCTCGGA |
| | TGGTAGTTGT | AGACACTCTG |
| | (SEQ ID NO: 1) | (SEQ ID NO: 2) |
| Runx2 | ACGCCATAGT | TCACTACCAG |
| | CCCTCCTTTT | CCACCGAGAC |
| | (SEQ ID NO: 3) | (SEQ ID NO: 4) |
| GAPDH | GTTGCTGTAGCC | GGTGGTCTCCTC |
| | AAATTCGTTGT | TGACTTCAACA |
| | (SEQ ID NO: 5) | (SEQ ID NO: 6) |

1.6 Immunoblotting

Cell lysates were prepared using M-PER© Protein Extraction Reagent (Pierce, Rockford, Ill., USA) plus protease inhibitor cocktails (Halt™; Pierce). Protein lysates (20-40 μg) were separated on 10% sodium dodecyl sulfate-polyacrylamide gel and transferred to polyvinylidene difluoride (PVDF) membranes. PVDF membranes were blocked in 5% blotting-grade milk in TBST (20 mM Tris-HCl [pH 7.6], 137 mM NaCl, 1% Tween 20). PVDF membranes were then probed with the indicated primary antibodies overnight at 4° C., washed, and then further probed with the corresponding secondary antibodies for 1 h. Immunoreactive proteins were detected by Western Bright Sirius Chemiluminescent Detection Reagent (Advansta, Menlo Park, Calif., USA) according to manufacturer's instructions. Images of chemiluminescent signals were captured using a LAS 3000 system (Fujifilm, Tokyo, Japan). The primary antibodies used were rat anti-TNAP antibody (1 μg/mL; R&D Systems, Minneapolis, Minn., USA), and rabbit anti-GAPDH antibody (1:10000; Gentex, Zeeland, Mich., USA) and mouse anti-beta-actin antibody (1:10000; ThermoFisher scientific). Horseradish peroxidase-conjugated secondary antibodies used were goat anti-rat IgG and anti-rabbit IgG (all at 1:5000 dilution and from Sigma-Aldrich). Immunoblotting experiments were done at least twice. All protein band densities were normalized to those of the GAPDH or beta-actin loading control.

1.7 Measurement of Intracellular ALP Activity

A fluorometric ALP Activity Detection kit (Abcam, Cambridge, UK) was used to measure intracellular ALP activity. Briefly, cells (5×10⁴) were homogenized in 100 μL assay buffer and 4-methylumbelliferyl phosphate disodium salt substrate was added. ALP cleaves the phosphate group of the non-fluorescent 4-methylumbelliferyl phosphate substrate, yielding fluorescent 4-methylumbelliferone. The stop solution was added after 30 min and the fluorescence intensity at excitation/emission wavelengths of 360/440 nm was measured using a Fluorescence Microtiter Plate Reader (Spectramax Gemini; Molecular Devices). ALP activity in samples was calculated using the following formula: ALP activity=A/V/T (mU/mL) where A is the amount of 4-methylumbelliferone generated by samples (nmol), V is the volume of sample added in the assay well (mL) and T is reaction time (min).

1.8 Lentiviral Vector-Mediated RNA Interference

The short-hairpin RNA (shRNA) expression plasmids and bacteria clones for TNAP (TRCN0000052005, target sequence: GCGCAAGAGACACTGAAATAT, (SEQ ID NO: 7)) and TRCN0000052007, target sequence: ACTGCCATCCTGTATGGCAAT, (SEQ ID NO: 8)), were obtained from the RNAi Core Facility, Academia Sinica (Taipei, Taiwan). The ALPL cDNA fragment was cut from plasmid obtained from the “Mammalian Gene Collection” of Genome Research Center, National Yang-Ming University (Clone: 066116) and amplified by PCR with Phusion High-Fidelity DNA polymerase (Thermo). The ALPL fragment was inserted into pLAS2w.Ppuro vector (RNAi Core Facility, Academia Sinica) by ligation at NheI and EcoRI sites (NEB). The procedure for preparing lentiviral vector and transduction was performed as previously described^{36,37}. Sub-confluent MSCs were transduced with lentiviral vector at a multiplicity of 5 in the presence of 8 μg/mL polybrene (Sigma-Aldrich). Twenty four hours later, the culture medium was replaced with fresh growth medium containing puromycin (1 μg/mL) to select the transduced cells for 48 h.

1.9 Immunofluorescence Staining

MSCs were cultured under osteogenic induction for 14 days, then were rinsed with PBS twice and fixed in 4% paraformaldehyde at room temperature for 20 min, followed by permeabilization with 0.1% Triton X-100 for 5 min at room temperature. Cells were then blocked by 1% bovine serum albumin in PBST for 1 h at room temperature. Mice anti-osteoadherin antibody (1:500 dilution, R&D) in blocking buffer was added for overnight incubation at 4° C. Cells were washed three times with PBS and then incubated with secondary antibody (goat anti-mice antibody conjugated with Alexa fluor-488) (1:400; Invitrogen) for 1 h at room temperature. Cell nuclei were counterstained with 4′, 6-diamidino-2-phenylindole (DAPI) (Sigma-Aldrich) in PBS. The fluorescent images were examined by a confocal microscope (Carl Zeiss LSM510) and captured by Zeiss EC Plan-Neofluar 40×/1.30 Oil DIC M27.

1.10 Immunohistochemistry Staining

BM (bone marrow) slices for immunohistochemistry staining were obtained from three AS patients (A1, A2, and A3), two healthy individuals (C4, C5) and one non-AS patient control (C6). Non-AS patient control was the one who received the bone marrow biopsy for lymphoma workup and was free of lymphoma. Among them, healthy controls (C4, C5) were Caucasians and their BM slides were purchased from US Biomax (Rockville, Md., USA). AS patients and non-AS patient control were Taiwanese. Their paraffin blocks of BM tissues were cut into 4-μm slices and processed using standard protocols. Sections were subjected to the treatment with Antigen Retrieval Reagent in Tris/EDTA (pH 9) solution (Thermo Fisher Scientific) and incubated with 0.1% Triton X-100 in phosphate-buffered saline for 15 min, followed by blocking with 3% human serum (Invitrogen, Carlsbad, Calif., USA) for 30 min. Sections were incubated with rabbit anti-TNAP antibody (1:200 dilution; Abcam) at 4° C. overnight, followed by incubation with horseradish peroxidase (HRP)-conjugated secondary antibody (Dako) for 1 h at room temperature. Brown color development was carried out by incubation with the chromogen 3, 3′-diaminobenzidine (Thermo Fisher Scientific). Nuclei were counterstained with hematoxylin (Thermo Fisher Scientific) as needed. Sections were washed in tap water for 5 min, dehydrated, and mounted with cover slips.

For double staining, after color development of the first staining with rabbit anti-TNAP antibody (1:200 dilution; Abcam) using HRP as described above, sections were then incubated with a second primary antibody: rabbit anti-MPO (1:1000 dilution overnight; DAKO) for myeloid lineages, mouse anti-CD68 (1:200 dilution for 3 h at room temperature; DAKO) for monocyte lineages, or rabbit anti-CD44 (1:200 dilution at 4° C. overnight; Abcam) for MSCs, followed by incubation with alkaline phosphatase-conjugated secondary antibody (1:500 dilution; SouthemBiotech) for 1 h at room temperature. Blue color development was carried out by incubation with the nitro blue tetrazolium chloride/5-bromo-4-chloro-3-indolyl phosphate, toluidine salt (Roche). Sections were washed in tap water for 5 min, dehydrated, and mounted with cover slips.

For identifying TNAP-positive cells, co-localization of TNAP/MPO, TNAP/CD68, and TNAP/CD44 positive cells was illustrated by using spectral imaging technique with MetaMorphic Offline version 7.8.2.0 software. After spectral unmixing using the spectral library (staining with TNAP antibody in brown; staining with another second antibody in blue), the composite pseudo-colored images were created, in which the co-localization of TNAP/MPO, TNAP/CD68, and TNAP/CD44 were represented as the turquoise color.

1.11 Animal Models

The animal experimental protocol was approved by the Animal Care and Utilization Committee of Taipei Tzu Chi General Hospital. NOD-SCID mice (8-10 weeks) were purchased from BioLASCO (Taipei, Taiwan) and maintained in a specific pathogen-free environment. Human MSCs were treated with β-glycerophosphate in culture for 5 days, embedded in fibrin (10⁶ cells in 5 μl fibrin) (Baxter, Vienna, Austria) and implanted in areas adjacent to right lamina of lumbar spine segments L4 to L5 in NOD-SCID mice. The lamina of lumbar spine was decorticated using the forceps. Mice were fed with a 0.9% phosphate diet (Dyets, Bethlehem, Pa., USA) after surgery. Micro-computed tomography (SkyScan 1076; Kontich, Belgium) over lumbar spines was undertaken 3 weeks after implantation. Bone mineral density over the femur measured by micro-computed tomography in NOD-SCID mice following implantation with AS MSCs and daily oral administration of H₂O levamisole (10 mg/kg), beryllium sulfate tetrahydrate (7.5 mg/kg), or pamidronate (0.3 mg/kg) for 12 weeks. Quantitative volumes of new bony apposition were acquired by Bruker CT-Volume version 2.0 software.

1.12 Measurements of Serum BAP Levels

Blood samples from human subjects were collected into EDTA-treated tubes. Serum was collected for measurement of levels of bone-specific TNAP (BAP) using the DiaSorin LIASON BAP OSTASE assay (one step delayed addition sandwich chemiluminescence immune assay) by the LIASON analyzer. The reference range of serum BAP level between 5.1-20.2 μg/L was considered normal according to the manufacturer.

1.13 Cytokine Release

MSCs were cultured under osteogenic induction for determining cytokine production. Supernatants were harvested at day 3, 7, and 10. Cytokines were detected using a Human Milliplex kit (Merck Millipore, Billerica, Mass., USA) according to manufacturer's instructions. Data were acquired by a Luminex 200 instrument (Luminex, Austin, Tex., USA) and analyzed by Milliplex analyst v5.1 (Merck Millipore).

1.14 Microarray Analyses

Total RNA was prepared from AS MSCs and N MSCs under osteogenic induction at days 0, 3 and 7 and used for human microarray chips (Affymetrix, Santa Clara, Calif., USA) at the GRC Microarray Core Facility, Academia Sinica. Sequential procedures of reverse transcription, biotin-conjugated nucleotide incorporation/fragmentation, and conversion of linear RNA into 35-200 nt biotin-labeled fragments using a GeneChip© 3′ IVT Express kit (Thermo Scientific, Waltham, Mass., USA) were undertaken. Then, samples were hybridized to a GeneChip Human Genome U133 Plus 2.0 array. Washing and staining of arrays were done using Fluidics Station 450 (Thermo Scientific), and array data were acquired with GeneChip Scanner 3000 (Thermo Scientific). Finally, array data were analyzed by GeneSpring GX v12 (Agilent Technologies, Santa Clara, Calif., USA).

1.15 Statistical Analyses

Data are shown as mean±standard error of mean (SEM) or mean standard deviation (SD). Data were analyzed by Student's t-test, Mann-Whitney U-test or Fisher's exact test. Correlations between variables were determined with the Spearman's rank correlation test. P<0.05 was considered significant. Data were analyzed by SPSS or Prism v6.01.

2. Results

2.1 the Pathologic Phenotype of Accelerated Mineralization in AS MSCs Under Osteogenic Induction is Runx2-Independent

We established an ex vivo cell culture model from BM-MSCs within tissues involved in spinal ankylosis of three AS patients (AS MSCs) to study the activation of the stromal progenitor cells in AS. We compared these cells with BM-MSCs of non-AS controls who underwent traumatic surgery (N MSCs). These cells adopted a fibroblastic-like morphology and expressed the characteristic surface markers of BM-MSCs. They possessed ti-lineage differentiation potentials as assayed by specific standard induction methods¹⁶.

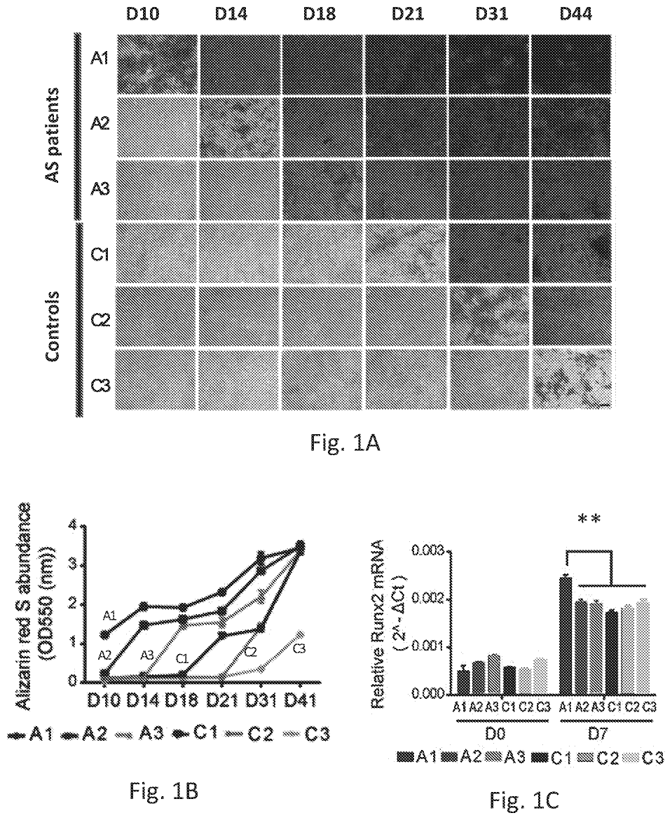

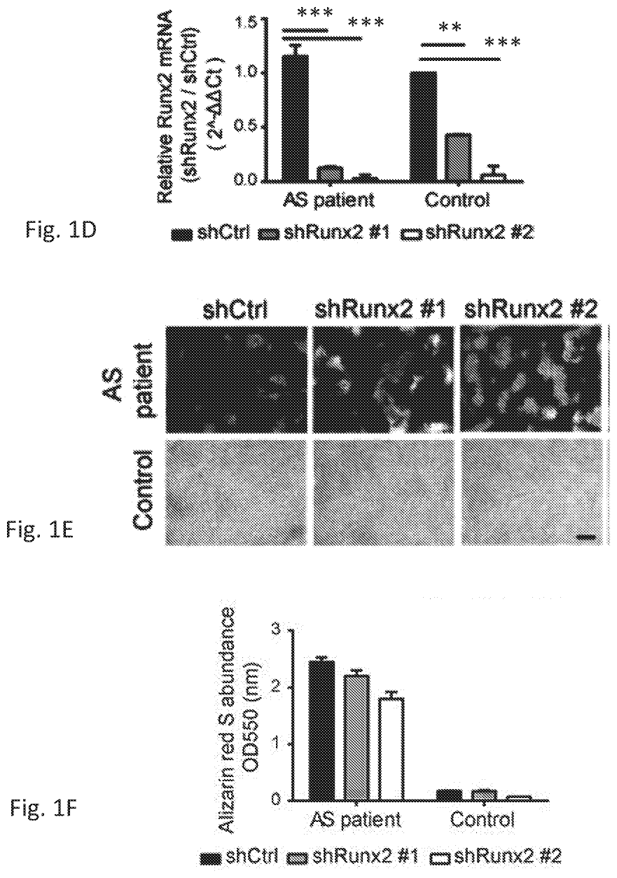

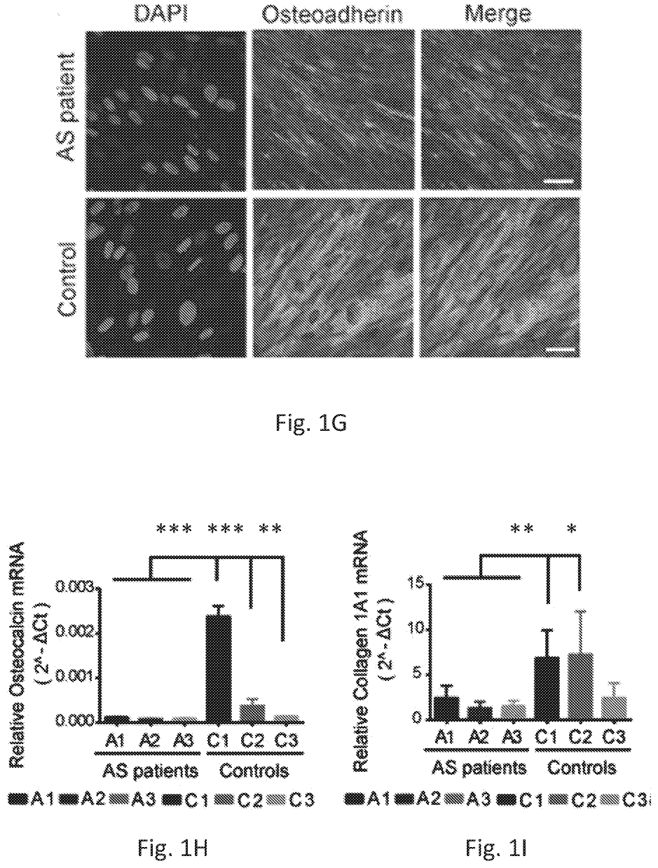

To assess the osteogenic potentials between AS MSCs and N MSCs, we cultured them in osteogenic induction media. Remarkably, staining with Alizarin Red S (ARS) for calcium deposition showed a significantly accelerated rate of mineralization in the three AS MSCs as compared with the three N MSCs (FIG. 1A, FIG. 1B). Generally, calcium deposition suggests differentiation of MSCs into osteoblasts that is regulated by the Runt-related transcription factor (Runx) 2¹⁷. It has been known that both Wingless (Wnt) 18 and bone morphogenetic protein (BMP)¹⁹ pathways are important for normal MSCs commitment to osteoblastogenesis through Runx2. However, we did not find the involvement of these pathways contributing to accelerated mineralization in either in vitro AS MSCs-culture or in vivo BM specimens of AS patients. In addition, though accelerated mineralization was found in AS MSCs, Runx2 expression was comparable between AS MSCs and N MSCs (FIG. 1C). Further, Runx2 knockdown in AS MSCs by shRNAs (FIG. 1D) did not affect the accelerated mineralization (FIG. 1E, FIG. 1F). Likewise, the expression of osteoblast markers, including oteoadherin, osteocalcin and collagen 1A1, was comparable between AS MSCs and N MSCs after osteogenic induction (FIGS. 1G-FIG. 1I). These results indicate that the abnormal mineralization, but not Runx-2 dependent osteoblastogenesis, contributes to the pathologic phenotype of AS MSCs after osteogenic induction.

2.2 Enhanced Expression of TNAP is Essential for Abnormal Mineralization in AS MSCs Under Osteogenic Induction

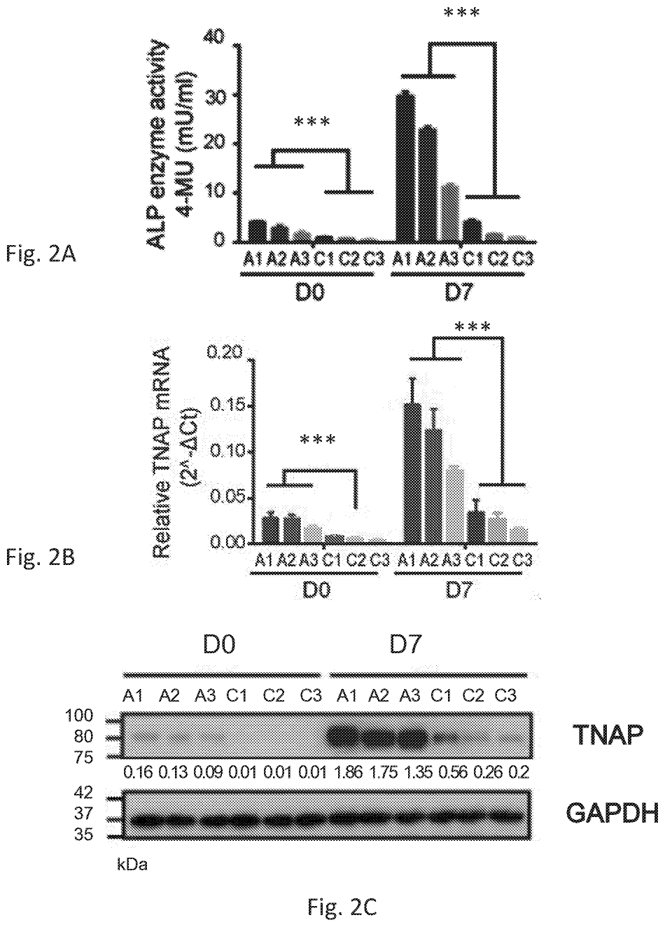

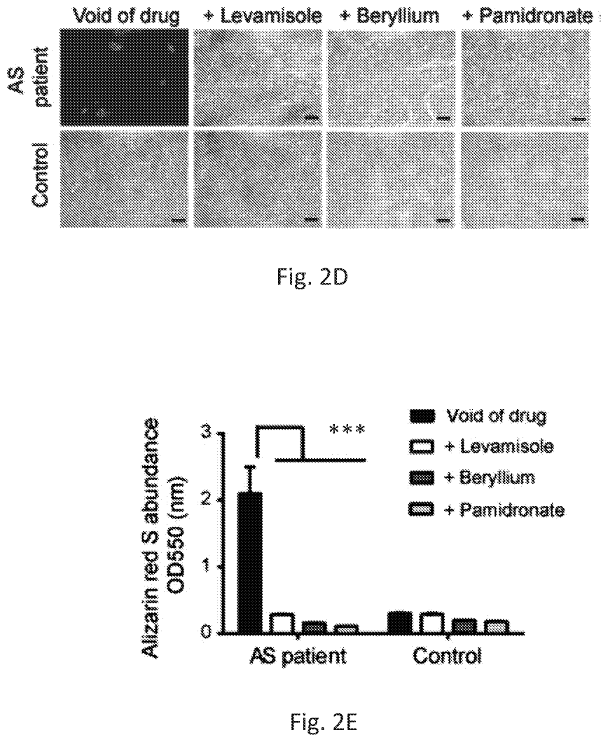

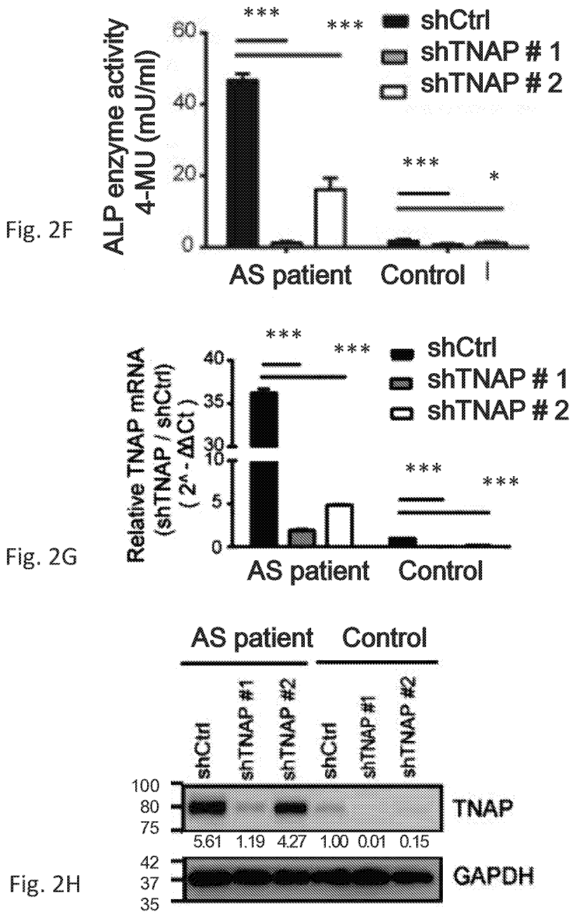

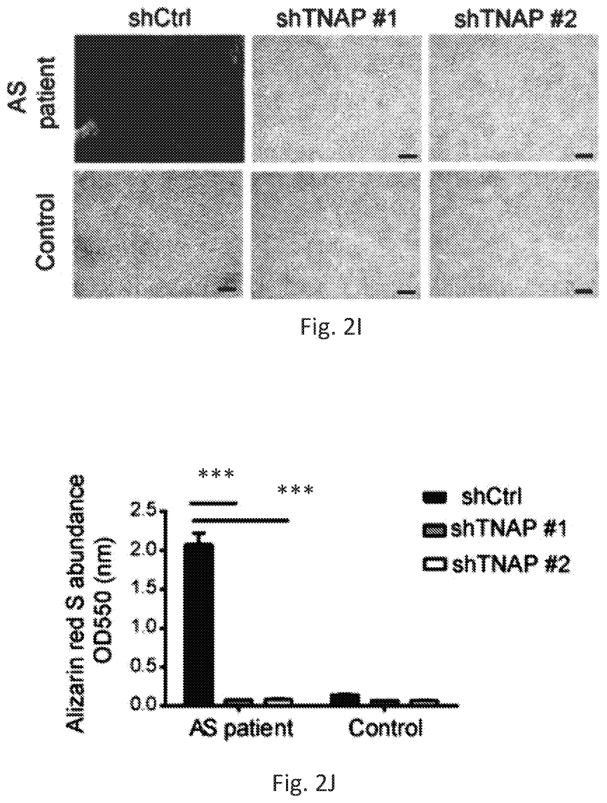

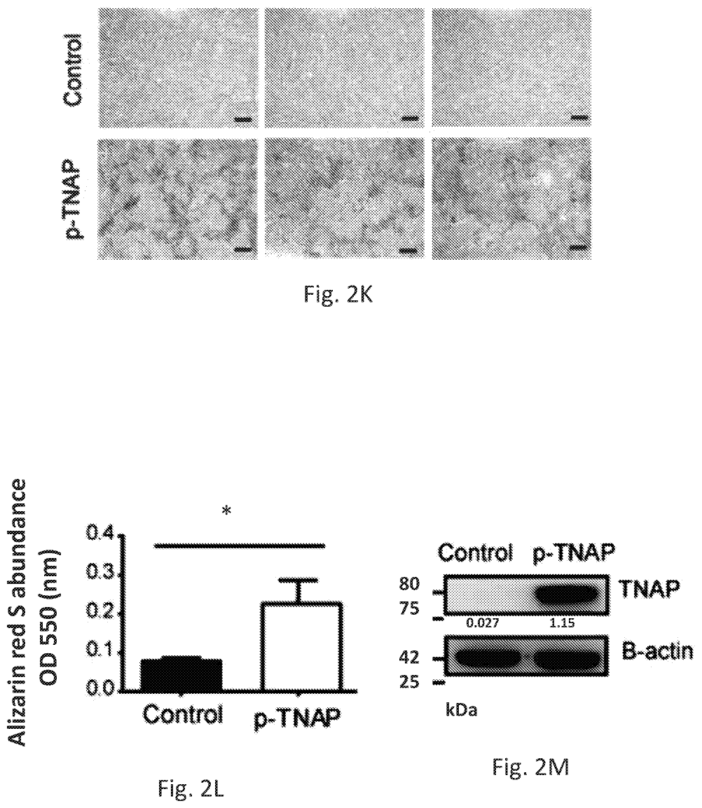

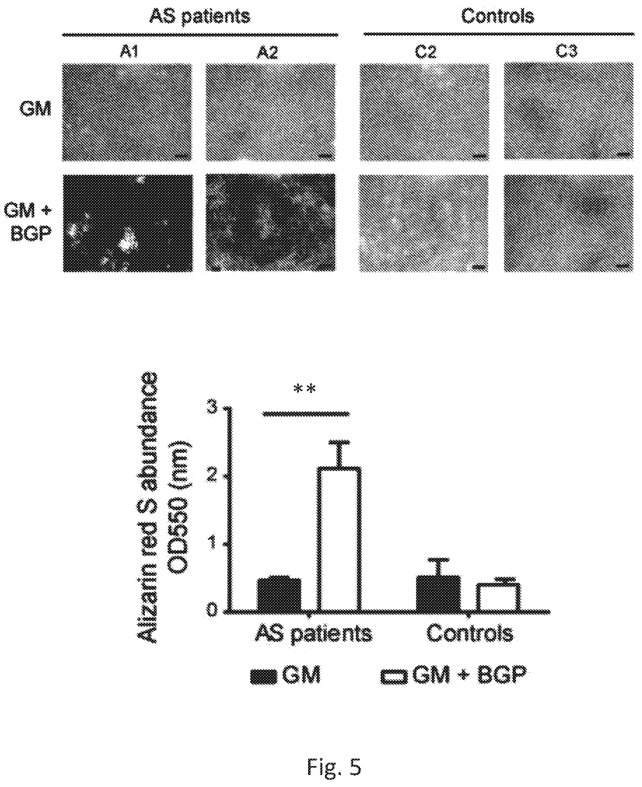

To further investigate the regulatory mechanism of accelerated mineralization of AS MSCs, we analyzed gene expression between AS MSCs and N MSCs after osteogenic induction at days 0, 3 and 7 by microarray analyses. One hundred and eighty-eight genes and 136 genes were up-regulated and down-regulated, respectively, more than two folds in AS MSCs, as compared with those in N MSCs, after osteogenic induction. Among these, the genes involved in osteogenesis were revealed by Ingenuity Pathway Analysis. Further validation of the genes involved in osteogensis by RT-QPCR revealed that elevation of tissue non-specific alkaline phosphatase (TNAP) expression and alkaline phosphatase (ALP) activity were most closely linked with the accelerated rate of differential mineralization in AS MSCs as compared with N MSCs, both before and after osteogenic induction. ALP is a large superfamily of unbiquitous ectoenzyme that can catalyze dephosphorylation and transphosphrylation reaction. It consists of four isoenzymes, including TNAP, placental, germ cell and intestinal ALP encoded by separate genes. Among them, TNAP is encoded by ALPL gene and distributed in liver/bone/kidney tissues with alternative splicing transcript variants^{20,21}. It hydrolyzes pyrophosphate and provides inorganic phosphate to promote mineralization^{20,21}. To determine the role of TNAP in abnormal mineralization of AS MSCs, we treated osteogenic cultures with uncompetitive (levamisole²² or pamidronate²³) and competitive (beryllium sulfate²⁴) TNAP inhibitors. Of note, accelerated mineralization in AS MSCs was blocked effectively by TNAP inhibitors (FIG. 2D, FIG. 2E). Similar reduction of accelerated mineralization in AS MSCs was observed when TNAP was silenced by two independent shRNAs against TNAP (FIGS. 2F-2J). Moreover, TNAP over-expression via lentiviral transduction in N MSCs showed enhanced mineralization (FIGS. 2K-2M). We further cultured MSCs in growth medium (GM) in the presence of β-glycerophosphate (the substrate of TNAP)²¹ (FIG. 5). As expected, N MSCs cultured in GM did not calcify. However, addition of β-glycerophosphate in AS MSCs showed a higher rate of mineralization than that in N MSCs, suggesting that increased expression of TNAP in AS MSCs was sufficient to induce accelerated mineralization in the presence of its substrate. Taken together, these results demonstrate that enhanced expression of TNAP is essential for accelerated mineralization in AS MSCs.

2.3 TNAP Blockade Inhibits New Bony Apposition Induced by AS MSCs in NOD-SCID mice

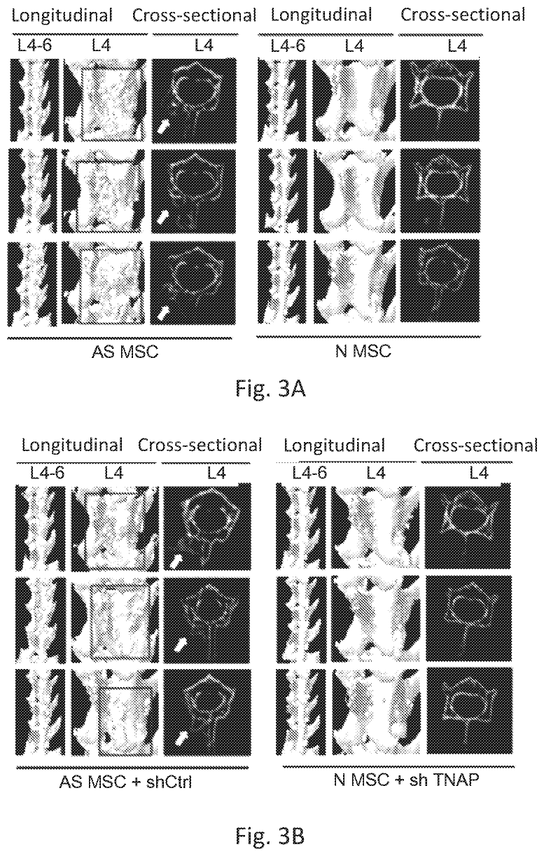

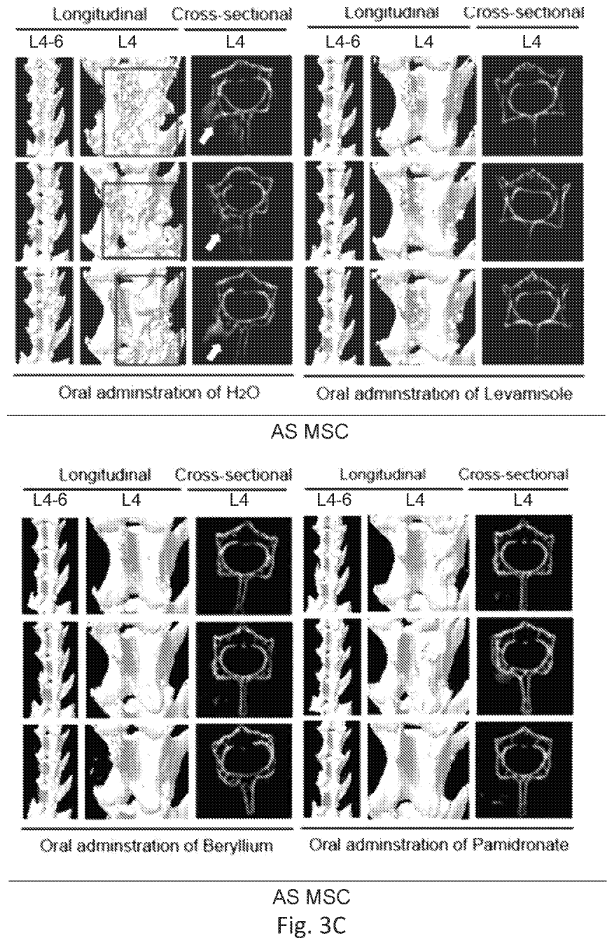

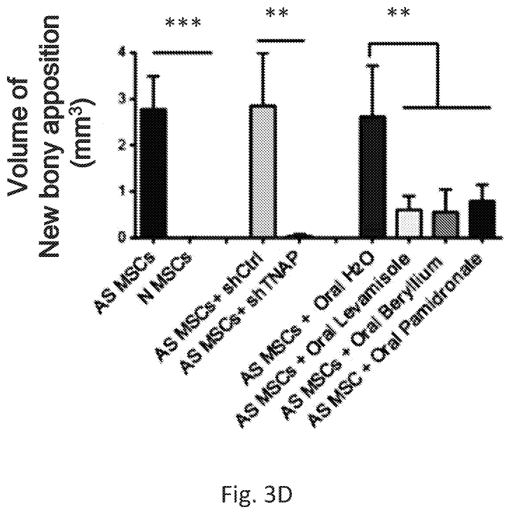

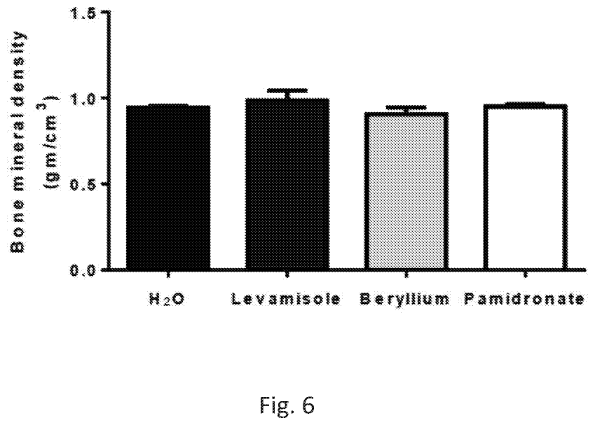

Next, we established an AS MSC-based in vivo disease model to mimic the pathological bony apposition, which could be used to test the therapeutic potential of TNAP inhibitors. Notably, NOD-SCID mice implanted with AS MSCs, but not N MSCs, adjacent to the right lamina of lumbar spine segment L4-5, developed new bony apposition (FIG. 3A). Furthermore, ectopic bony apposition induced by implantation of AS MSCs was blocked if TNAP was silenced by shRNA as compared with control shRNA-transduced AS MSCs (FIG. 3B). Consistently, oral administration of TNAP inhibitors largely abrogated new bony apposition in NOD-SCID mice implanted with AS MSCs (FIG. 3C), but did not alter overall bone mineral density (BMD) (FIG. 6). The quantitative volumes of new bony apposition between groups were shown (FIG. 3D). These results suggest that TNAP blockade could inhibit new bony apposition induced by AS MSCs in NOD-SCID mice.

2.4 Serum Bone-Specific TNAP Levels are Significantly Associated with Radiographic Severity in AS Patients and have the Potential to be the Prognostic Marker

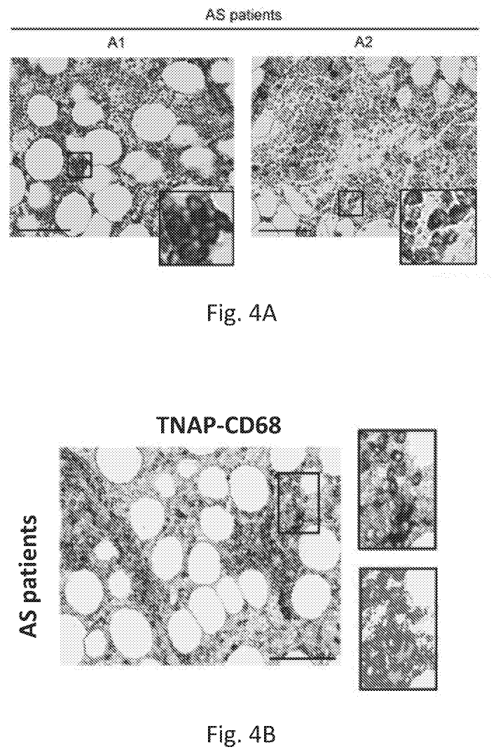

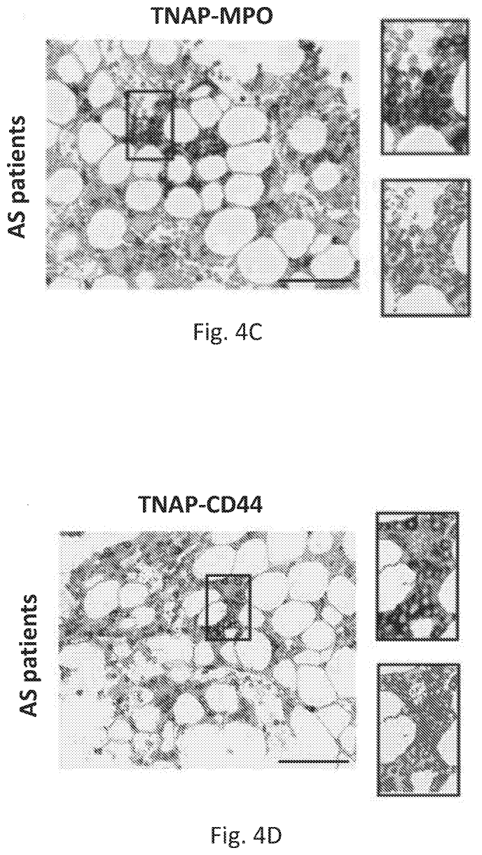

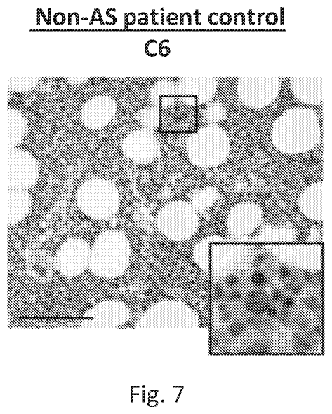

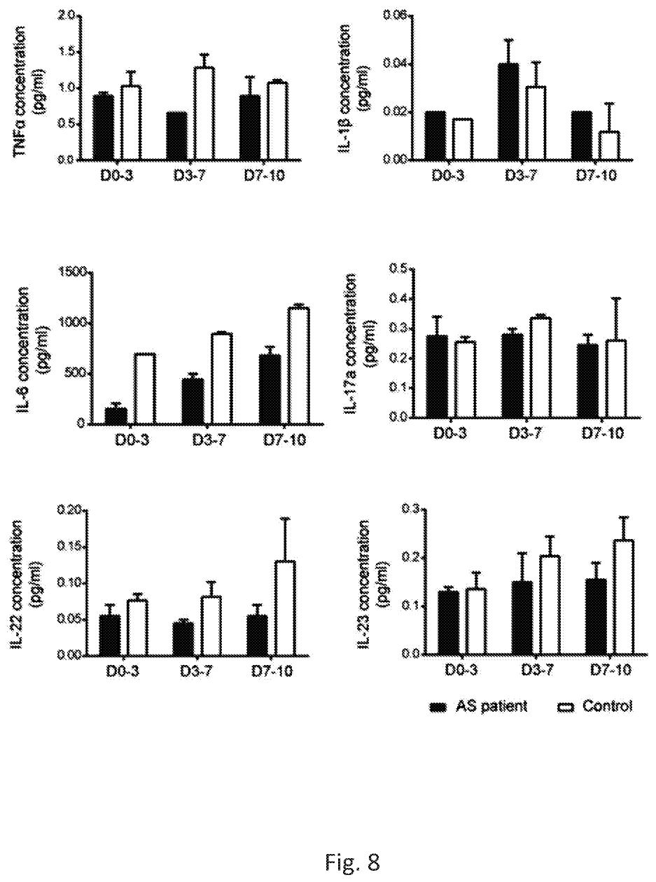

Next, we wondered if enhanced expression of TNAP can be detected in the BM or peripheral blood of AS patients. Immunohistochemistry (IHC) staining of the BM specimen revealed increased expression of TNAP in the BM of AS patients, as compared with healthy individuals (FIG. 4A) and non-AS patient control (FIG. 7). IHC double staining showed most TNAP-positive cells were myeloid (myeoperoxidase⁺), monocyte (CD68⁺) lineages or MSCs (CD44⁺) (FIGS. 4B, 4C, 4D). In addition, instead of serum TNAP, we measured serum levels of bone-specific TNAP (BAP) to exclude the interference of TNAP from liver and kidney using samples from two separate cohorts: one is a Taiwanese cohort which included 104 AS patients and 50 healthy controls, the other is a British cohort with 184 AS patients. Their demographic characteristics and the comparative difference between subgroups/cohorts were shown (Table 1-3). The percentages of AS patients with increased serum BAP levels (>20.2 μg/L) in Taiwanese and British cohorts were 9.61% and 25.54%, respectively. Overall, serum BAP levels were significantly higher in AS patients than healthy controls (12.954±5.538 μg/L and 6.296±2.110 μg/L; P<0.001) in the Taiwanese cohort, while serum BAP levels were 17.607±8.055 μg/L in the British cohort

TABLE 1
Characteristics of AS patients with normal and increased BAP levels in “Taiwanese cohort”

| Characteristics | Total AS | | Patients with | | Patient with | | |
|---|---|---|---|---|---|---|---|
| (Patient number with | patients | | normal BAP | | increased BAP | | |
| normal/increased BAP) | (n = 104) | | (n = 94) | | (n = 10) | | P value |
| Male/Female (94/10) | 90/14 | | 81/13 | | 9/1 | | 1.000 |
| Age (y/o) (94/10) | 46.000 | (12.152) | 45.745 | (11.997) | 48.400 | (13.986) | 0.440 |
| Onset age (y/o) (92/10) | 27.029 | (10.380) | 27.065 | (10.309) | 26.700 | (11.596) | 0.791 |
| Disease duration (years) (92/10) | 19.128 | (11.163) | 18.848 | (11.054) | 21.700 | (12.437) | 0.558 |
| HLA-B27 positive/negative (92/9) | 95/6 | | 86/6 | | 9/0 | | 1.000 |
| Serum BAP (μg/L) (94/10) | 12.954 | (5.538) | 11.640 | (3.846) | 25.300 | (3.287) | <0.001* |
| ESR (mm/hour) (91/10) | 13.386 | (12.751) | 13.528 | (13.305) | 12.100 | (5.859) | 0.677 |
| CRP (mg/dl) (92/10) | 0.715 | (1.030) | 0.734 | (1.070) | 0.538 | (0.551) | 0.897 |
| BASDAI (93/10) | 2.680 | (1.826) | 2.698 | (1.855) | 2.520 | (1.603) | 0.902 |
| BASFI (92/10) | 1.108 | (1.411) | 1.055 | (1.416) | 1.600 | (1.323) | 0.119 |
| BAS-G (94/10) | 3.421 | (2.782) | 3.383 | (2.870) | 3.775 | (1.805) | 0.340 |
| mSASSS (89/10) | 31.051 | (18.135) | 29.876 | (17.886) | 41.500 | (17.834) | 0.047* |
| BASRI-Total (89/10) | 9.823 | (2.591) | 9.534 | (2.358) | 12.400 | (3.239) | 0.009* |
| BASRI-Hip (90/10) | 0.540 | (1.171) | 0.400 | (0.998) | 1.800 | (1.814) | 0.003* |
| BASRI-SI (89/10) | 3.308 | (0.609) | 3.264 | (0.608) | 3.700 | (0.483) | 0.031* |
| BASRI-C (89/10) | 2.838 | (0.792) | 2.787 | (0.776) | 3.300 | (0.823) | 0.058 |
| BASRI-L (89/10) | 3.111 | (0.925) | 3.056 | (0.922) | 3.600 | (0.843) | 0.053 |

Values are shown as mean (standard deviation: SD.).
P is determined by Mann-Whitney U or Fisher exact test between AS patients with normal and increased BAP.
*Statistically significant.

TABLE 2
Characteristics of AS patients with normal and increased BAP levels in “British cohort”

| Characteristics | Total AS | | Patients with | | Patient with | | |
|---|---|---|---|---|---|---|---|
| (Patient number with | patients | | normal BAP | | increased BAP | | |
| normal/increased BAP) | (n = 184) | | (n = 137) | | (n = 47) | | P value |
| Male/Female (137/47) | 136/48 | | 98/39 | | 38/9 | | 0.209 |
| Age (y/o) (137/47) | 53.321 | (13.280) | 53.066 | (13.430) | 54.064 | (12.945) | 0.612 |
| Disease duration (years) (137/47) | 21.668 | (13.346) | 21.883 | (13.249) | 21.043 | (13.752) | 0.738 |
| HLA-B27 positive/negative (137/47) | 162/22 | | 123/14 | | 39/8 | | 0.215 |
| Serum BAP (μg/L) (137/47) | 17.607 | (8.055) | 14.300 | (3.555) | 27.245 | (9.656) | <0.001* |
| CRP (mg/dl) (137/47) | 1.104 | (1.784) | 0.989 | (1.617) | 1.442 | (2.184) | 0.027* |
| BASDAI (137/47) | 3.666 | (2.063) | 3.515 | (2.036) | 4.109 | (2.100) | 0.110 |
| BASFI (137/47) | 3.889 | (2.382) | 3.518 | (2.169) | 4.972 | (2.657) | <0.001* |
| BAS-G (137/47) | 3.783 | (2.438) | 3.591 | (2.381) | 4.340 | (2.539) | 0.068 |
| mSASSS (137/47) | 19.630 | (22.191) | 17.964 | (22.083) | 24.489 | (22.019) | 0.036* |

Values are shown as mean (SD.).
P value is determined by Mann-Whitney U test or Fisher exact test between AS patients with normal and increased BAP.
*Statistically significant.

TABLE 3
Demography of AS patients and healthy controls
in “Taiwanese cohort”

| | Total AS patients | | Healthy controls | | |
|---|---|---|---|---|---|
| Characteristic | (n = 104) | | (n = 50) | | P values |
| Male/Female | 90/14 | | 32/18 | | 0.003* |
| Age (y/o) | 46.00 | (12.152) | 42.94 | (14.649) | 0.203 |
| Serum BAP (μg/L) | 12.954 | (5.538) | 6.296 | (2.110) | <0.001* |

Values are shown as mean (SD.).
P value is determined by Mann-Whitney U test or Fisher exact test.
*Statistically significant.

We then tested if serum BAP levels are correlated with clinical parameters. Radiographic severity was measured by the Bath Ankylosing Spondylitis Radiology Index-total score (BASRI-total) or modified Stoke Ankylosing Spondylitis Spinal Score (mSASSS). Remarkably, there were significant positive correlations between serum BAP levels and radiographic severity in AS patients (P<0.05 for both cohorts by mSASSS; P<0.001 for the Taiwanese cohort by BASRI-total; BASRI-total was not recorded for the British cohort) (Table 4a). Likewise, for clarifying risk factors that could be used to predict radiographic severity in AS patients, multivariate regression analyses were performed using various models. These results showed that serum BAP levels, disease duration and CRP were independent risk factors in Taiwanese cohort; while in British cohort, serum BAP levels, disease duration and male gender were independent risk factors (Table 4b). Of note, serum BAP levels and disease duration are the two common predictors regardless of the different models of analysis. Next, we analyzed the data from 37 AS patients who had longitudinal follow-up of radiographic change retrospectively in Taiwanese cohort (Table 5). In this longitudinal AS subgroup analysis, we found serum BAP levels and CRP were two independent risk factors for prediction of yearly radiographic progression in AS patients (Table 4c). To summarize, the analyses of both Taiwanese and British cohorts demonstrated that the serum BAP level may be a prognostic biomarker for AS patients at high risk of spinal ankylosis.

Table 4a. The correlation analysis between serum BAP concentrations and clinical parameters by Spearman's rank correlation test.

TABLE 4a
Correlations between serum levels of BAP and
clinical parameters of AS patients in “Taiwanese
cohort” and “British cohort”

| | Serum BAP levels | | | |
|---|---|---|---|---|
| | Taiwanese cohort | | British cohort | |
| Variables | (n = 104) | | (n = 184) | |
| Age (years old) | 0.143 | (0.149) | 0.050 | (0.503) |
| Onset age (years old) | 0.181 | (0.069) | N.R. | |
| Disease duration (years) | −0.025 | (0.805) | −0.025 | (0.735) |
| ESR (mm/hour) | −0.021 | (0.834) | N.R. | |
| CRP (mg/dl) | 0.065 | (0.514) | 0.205 | (0.005 *) |
| BASDAI | −0.035 | (0.723) | 0.121 | (0.103) |
| BASFI | 0.10 | (0.316) | 0.227 | (0.002 *) |
| BAS-G | 0.131 | (0.186) | 0.159 | (0.031 *) |
| mSASSS | 0.221 | (0.028^{A}) | 0.185 | (0.012 *) |

Values are shown as r (P value); r: Spearman's correlation coefficiency.
N.R.: not recorded.
* Significantly difference.

Table 4b. The multivariate regression analysis for assessing predictors of radiographic severity (mSASSS) in AS patients of “Taiwanese cohort” and “British cohort”

TABLE 4b
Multivariate linear regression analysis for prediction of radiographic
severity in AS patients of “Taiwanese and British cohorts”

| | Regression coefficient | | Standard | |
|---|---|---|---|---|
| Models | (95% CI) | | coefficient | P value |
| Taiwanese cohort | | | | |
| Model 1: mSASSS | | | | |
| Serum BAP | 0.628 | (0.049 to 1.206) | 0.191 | 0.034 * |
| CRP | 3.473 | (0.332 to 6.615) | 0.195 | 0.031 * |
| Disease duration | 0.760 | (0.468 to 1.052) | 0.474 | <0.001 * |
| Gender | −3.020 | (−12.695 to 6.655) | −0.057 | 0.537 |
| Adjusted R² | 0.271 | | | |
| British cohort | | | | |
| Model 2: mSASSS | | | | |
| Serum BAP | 0.464 | (0.104 to 0.825) | 0.169 | 0.012 * |
| CRP | 0.561 | (−1.060 to 2.182) | 0.045 | 0.495 |
| Disease duration | 0.617 | (0.402 to 0.832) | 0.371 | <0.001 * |
| Gender | −11.493 | (−18.064 to −4.922) | −0.228 | 0.001 * |

| | |
|---|---|
| Adjusted R² | 0.216 |

Gender: 1 = male, 2 = female.
CI = confidence interval.
* Statistically significant.

Table 4c. The multivariate regression analysis for assessing predictors of yearly radiographic progression in a longitudinal AS subgroup of “Taiwanese cohort”.*

TABLE 4c
Multivariate linear regression analysis for prediction
of “yearly radiographic change” in longitudinal
AS subgroup of “Taiwanese cohort”

| Model | Regression | | | |
|---|---|---|---|---|
| Yearly change | coefficient | | Standard | |
| in mSASSS | (95% CI) | | coefficient | P value |
| Serum BAP | 0.094 | (0.005 to 0.183) | 0.306 | 0.040 * |
| CRP | 0.719 | (0.398 to 1.040) | 0.614 | <0.001 * |
| Gender | −1.051 | (−2.833 to 0.730) | −0.170 | 0.238 |

| | |
|---|---|
| Adjusted R² | 0.362 |

Gender: 1 = male, 2 = female.
CI = confidence interval.
* Statistically significant

TABLE 5
Demography of a longitudinal AS subgroup with retrospective follow-up
of radiographic progression in “Taiwanese cohort”

| Characteristic | AS patients (n = 37 ) | |
|---|---|---|
| Male/Female | 34/3 | |
| Age (y/o) | 52.108 | (10.498) |
| Disease duration (years) | 23.946 | (11.891) |
| Interval of longitudinal | 5.730 | (2.832) |
| radiographic follow-up (years) | | |
| ESR (mm/hour) | 14.784 | (15.013) |
| CRP (mg/dl) | 0.953 | (1.466) |
| BASDAI | 2.730 | (1.859) |
| BASFI | 1.761 | (1.682) |
| BAS-G | 4.000 | (2.967) |
| mSASSS | 40.892 | (18.403) |
| Yearly change in mSASSS | 1.758 | (1.715) |
| BASRI-Total | 11.216 | (2.335) |
| Yearly change in BASRI-Total | 0.243 | (0.352) |
| Serum BAP (pg/L) | 13.949 | (5.608) |

Values are shown as mean (SD.).

3. Summary

We here established mesenchymal stromal cells from bone marrow of entheseal tissues of ankylosis spine of AS patients (AS MSC) after spinal osteotomy, which can be used for studying the stromal progenitor cell activation during osteogenesis and for the potential cell-based platform to identify drugs for solving ankylosis of AS patients. The AS MSCs derived in this invention show the abnormal phenotype of accelerated mineralization after osteogenic induction, of which tissue non-specific alkaline phosphatase (TNAP) activity was highly expressed. The AS MSCs derived in this invention can induce ectopic bony apposition in animals when implanted into spine of NOD-SCID mice, serving as a good animal model.

Importantly, blockage of TNAP via its non-competitive chemical inhibitors, such as levamisole or nitrogen-containing bisphosphates (such as pamidronate), would inhibit this abnormal mineralization of AS MSCs in culture. The ectopic bony apposition induced by AS MSCs implantation in NOD-SCID mice would be blocked after oral administration of levamisole or pamidronate. Of interest, levamisole has been used as an anthelmintic agent for several years²⁵. Some studies have reported that levamisole can marginally reduce inflammation of AS patients with the proposed action on T regulatory cells²⁶⁻²⁹. However, its clinical application in blocking syndesmphyte formation through TNAP inhibition has not been explored. The long-treatment of levamisole did not affect bone density of treated mice in our experiments. In addition, another critical prospect would be to develop a dual-purpose drug with both effects on TNAP inhibition and osteoporosis prevention, of which nitrogen-containing bisphosphates (such as pamidronate) is worthy of exploration²³. While bisphosphates have been widely used in osteoporosis treatment via inhibiting osteoclastic bone resorption, they can also inhibit TNAP enzyme through chelation of Mg²⁺/Zn²⁺ with their bone hook structure²³. Interestingly, our data identified that pamidronate not only blocks enhanced mineralization of AS MSCs under osteogenic induction, but also inhibits new bony apposition in our AS-MSCs-based animal model. Although the clinical application of pamidronate has been proposed in control of AS disease activity through its anti-inflammatory effects³⁰⁻³⁵, such as modulation of pro-inflammatory cytokines in macrophage/monocyte lineages, only one study suggested its effect on lowering serum BAP levels³⁴. In the prior art, there is no available data indicating its effect on blocking syndesmophyte formation.

Therefore, the AS MSCs established here could serve as a useful platform for exploring the effect of potential therapeutic treatment for resolving spinal fusion problem in AS patients in the future.

We also demonstrated that high serum TNAP/or bone specific ALP (BAP) levels are significantly correlated with the radiographic severity of AS patients, which would be a potential prognosis biomarker for predicating high risk of AS patients with spinal ankylosis in the future. This finding could be very helpful for the clinical physicians to early identify high-risk group of AS patients with potentially easy x-ray progression, further combined with early treatment via oral levamisole or selective TNAP inhibitor would prospect a potential efficacy to arrest spinal ankylosis of AS patients.

### REFERENCE

1. Dougados, M. et al. Spondyloarthritis. Lancet. 377, 2127-2137 (2011).
2. Tam, L. S. et al. Pathogenesis of ankylosing spondylitis. Nat. Rev Rheumatol. 6, 399-405 (2010).
3. Wanders, A. et al. Nonsteroidal antiinflammatory drugs reduce radiographic progression in patients with ankylosing spondylitis: a randomized clinical trial. Arthritis Rheum. 52, 1756-1765 (2005).
4. van der Heijde, D. et al. Radiographic progression of ankylosing spondylitis after up to two years of treatment with etanercept. Arthritis Rheum. 58, 1324-1331 (2008).
5. van der Heijde, D. et al. Radiographic findings following two years of infliximab therapy in patients with ankylosing spondylitis. Arthritis Rheum. 58, 3063-3070 (2008).
6. van der Heijde, D. et al. Assessment of radiographic progression in the spines of patients with ankylosing spondylitis treated with adalimumab for up to 2 years. Arthritis Res Ther. 11, R127 (2009).
7. Braun, J. et al. The effect of two golimumab doses on radiographic progression in ankylosing spondylitis: results through 4 years of the GO-RAISE trial. Ann Rheum Dis. 73, 1106-1113 (2013).
8. Baraliakos, X. el al. Continuous long-term anti-TNF therapy does not lead to an increase in the rate of new bone formation over 8 years in patients with ankylosing spondylitis. Ann Rheum Dis. 73, 710-715 (2013).
9. Maas, F. et al. Reduction in spinal radiographic progression in ankylosing spondylitis patients receiving prolonged treatment with tumor necrosis factor inhibitors. Arthritis Care Res (Hoboken). 69, 1011-1019 (2017).
10. van der Heijde, D. et al. MRI-inflammation of the vertebral unit (VU) only marginally contributes to new syndesmopyhte formation in that unit: a multi-level-analysis. Ann. Rheum. Dis. 67(Suppl. II), 130 (2007).
11. Lories, R. J. et al. Evidence for uncoupling of inflammation and joint remodeling in a mouse model of spondylarthritis. Arthritis Rheum. 56, 489-497 (2007)
12. Baraliakos, X. et al. The relationship between inflammation and new bone formation in patients with ankylosing spondylitis. Arthritis Res. Ther. 10, R104 (2008).
13. Xie, Z. et al. MCP1 triggers monocyte dysfunctions during abnormal osteogenic differentiation of mesenchymal stem cells in ankylosing spondylitis. J Mol Med (Berl). 95, 143-154 (2017).
14. Xie, Z. et al. Imbalance between bone morphogenetic protein 2 and noggin induces abnormal osteogenic differentiation of mesenchymal stem cells in ankylosing spondylitis. Arthritis Rheumatol. 68, 430-440 (2016).
15. Prockop, D. J. Marrow stromal cells as stem cells for nonhematopoietic tissues. Science. 276, 1-4 (1997).
16. Dominici, M. et al. Minimal criteria for defining multipotent mesenchymal stromal cells. The International Society for Cellular Therapy position statement. Cytotherapy. 8, 315-17 (2006).
17. Karsenty, G. Transcriptional control of skeletogenesis. Annu Rev Genomics Hum Genet. 9, 183-196 (2008).
18. Kim, J. H. et al. Wnt signaling in bone formation and its therapeutic potential for bone disease. Ther Adv Musculoskelet Dis. 5, 13-31 (2013).
19. Chen, G. et al. TGF-β and BMP signaling is osteoblast differentiation and bone formation. Int J Biol Sci. 8, 272-288 (2012).
20. Orimo, H. The mechanism of mineralization and the role of alkaline phosphatase in health and disease. J Nippon Med Sch. 77, 4-12 (2010).
21. Millin, J. L. et al. Alkaline phosphatases: Structure, substrate specificity and functional relatedness to other members of a large superfamily of enzymes. Purinergic Signal. 2, 335-341 (2006).
22. Kozlenkov, A. et al. Residues determining the binding specificity of uncompetitive inhibitors to tissue-nonspecific alkaline phosphate. J Bone Miner Res. 19, 1862-1872 (2004).
23. Vaisman, D. N. et al. Bone-specific alkaline phosphatase activity is inhibited by bisphosphonate: role of divalent cations. Biol Trace Elem Res. 104, 131-140 (2005).
24. Price, P. A. et al. Tissue-nonspecific alkaline phosphatase is required for the calcification of collagen in serum: a possible mechanism for biomineralization. J Biol Chem. 284, 4594-4604 (2009).
25. Janseen, P. A. The levamisole story. Jucker E (ed). Progess in Drug Research. New York, Springer-Verlag. 1-66 (1976)
26. Rosenthal M. A critical review of the effect of levamisole in rheumatic diseases other than rheumatoid arthritis. J Rheumatol. Suppl. 4, 97-100 (1978).
27. Rosenthal, M. et al. Immunotherapy with levamisole in rheumatic diseases. Scand J Rheumatol. 5, 216-220 (1976).
28. Christensen, K. D. Treatment of seronegative spondylarthritis with levamisole: A double-blind placebo-controlled study. Int J Immunopharmacol. 1, 147-150 (1979).
29. Goldstein G. Mode of action of levamisole. J Rheumatol. Suppl. 4, 143-148 (1978).
30. Toussirot, E. et al. Anti-inflammatory treatment with bisphosphates in ankylosing spondylitis. Curr Opin Rheumatol. 19, 340-345 (2007).4
31. Toussirot, E. el al. Transient efficacy of pulse pamidronate treatment in active spondylarthropathies: an open study of 35 cases. Clin Exp Rheumatol. 24, 348 (2006).
32. Santra, G. et al. Assessment of the efficacy of pamidronate in ankylosing spondylitis: an open prospective trial. Singapore Med J. 51, 883-887 (2010).
33. Carins, A. P. el al. An open study of pulse parmidronate treatment in severe ankylosing spondylitis, and its effect on biochemical markers of bone turnover. Ann Rheum Dis. 64, 338-339 (2005).
34. Kang, K. Y et al. Increased serum alkaline phosphatase levels correlate with high disease activity and low bone mineral density in patients with axial spondyloarthritis. Semin Arthritis Rheum. 45, 202-207 (2015).
35. Maksymowych, W. P. et al. A six-month randomized, controlled, double-blind, dose-response comparison of intravenous pamidronate (60 mg versus 10 mg) in the treatment of nonsteroidal antiinflammatory drug-refractory ankylosing spondylitis. Arthritis Rheum. 46, 766-773 (2002).
36. Lin, K. I., Calame K. Introduction of genes into primary murine splenic B cells using retrovirus vectors. Methods Mol Biol. 271, 139-418 (2004).
37. Lin F. R. el al. Introduction of apoptosis in plasma cells by B lymphocyte-induced maturation protein-1 knockdown. Cancer Res. 67, 11914-11923 (2007).

1. A method for detecting ankylosing spondylitis (AS) and/or predicting the risk of development of radiographic severity of AS, comprising
(i) providing a biological sample from a subject; and
(ii) detecting an ALPL gene product as an AS marker in the sample.

2. The method of claim 1, wherein the gene product includes a protein or a RNA transcript.

3. The method of claim 1, wherein the ALPL gene product is a non-specific alkaline phosphatase (TNAP).

4. The method of claim 1, wherein the ALPL gene product is a bone-specific TNAP (BAP).

5. The method of claim 1, wherein the marker is detected with an agent that specifically binds to the ALPL gene product.

6. The method of claim 1, wherein the detection is performed by an immunoassay, a mass spectrometric assay, a nucleic acid hybridization detection assay, and/or a reverse transferase-polymerase chain reaction (RT-PCR).

7. The method of claim 1, wherein the biological sample is a body fluid sample or a tissue sample.

8. The method of claim 1, comprising comparing the results of the detection with a reference level and identifying the subject as having AS and/or at risk of development of radiographic severity of AS, if the comparison shows an elevated level of the ALPL gene product.

9. The method of claim 8, further comprising applying a further AS diagnostic assay to the subject to confirm AS occurrence or the risk of development of radiographic severity of AS.

10. The method of claim 8, wherein the radiographic severity includes one or more radiographic features of AS selected from the group consisting of erosion, sclerosis, squaring, syndesmophyte formation, bony bridge, spinal ankylosing and any combination thereof.

11. The method of claim 8, further comprising treating the subject with anti-AS therapeutic methods and/or medicaments.

12. The method of claim 11, wherein the anti-AS therapeutic methods include surgery and/or physical therapy, and the medicaments include corticosteroids, non-steroidal anti-inflammatory drugs (NSAISs), an immunosuppressant, a tumor necrosis factor-alpha (TNF-α) blocker inhibitor, an anti-interleukin-6 inhibitor, an interleukin 17A inhibitor, and/or a TNAP inhibitor.

13. A method for monitoring progression of AS in an AS patient, comprising
(a) providing a first biological sample from the patient at a first time point,
(b) providing a second biological sample from the patient at a second time point, which is later than the first time point,
(c) measuring the levels of a ALPL gene product as an AS marker in the first and second biological samples; and
(d) determining AS progression in the patient based on the levels of ALPL gene product in the first and second biological samples, wherein an elevated level of ALPL gene product in the second biological sample as compared to that in the first biological sample is indicative of AS progression.

14. The method of claim 13, wherein the AS progression includes an increased level of radiographic severity of AS.

15. The method of claim 14, wherein the radiographic severity includes one or more radiographic features of AS selected from the group consisting of erosion, sclerosis, squaring, syndesmophyte formation, bony bridge, spinal ankylosing and any combination thereof.

16. A method for treating AS, comprising administering a therapeutically effective amount of a TNAP inhibitor to a subject in need.

17. The method of claim 16, wherein the method is effective in reducing or alleviating one or more conditions of AS.

18. The method of claim 17, wherein the AS conditions include radiographic severity.

19. The method of claim 18, wherein the radiographic severity includes one or more radiographic features of AS selected from the group consisting of erosion, sclerosis, squaring, syndesmophyte formation, bony bridge, spinal ankylosing and any combination thereof.

20. The method of claim 16, wherein the TNAP inhibitor is selected from the group consisting of levamisole, beryllium sulfate tetrahydrate, pamidronate or any combination thereof.

21. The method of claim 16, wherein the TNAP inhibitor is administered in combination with anti-AS therapeutic methods and/or other medicaments.

22.-29. (canceled)

30. A method for producing a non-human animal model for AS, comprising the steps of
(i) providing marrow mesenchymal stem cells (BMSCs) from bone marrow in the spine afflicted with spinal ankyloses from a AS patient;
(ii) implanting the BMSCs in areas adjacent of lamina of lumbar spine segments in an immunodeficient non-human animal; and
(iii) growing the animal in a condition suitable to induce one or more symptoms/conditions of AS.

31. The method of claim 30, wherein the lumbar spine segments is L4 to L5, and/or the AS symptoms/conditions include radiographic severity.

32. The method of claim 31, wherein the radiographic severity includes one or more radiographic features of AS selected from the group consisting of erosion, sclerosis, squaring, syndesmophyte formation, bony bridge, spinal ankylosing and any combination thereof.

33. A non-human animal model for AS prepared by the method of claim 30.

34. A method for screening for an agent effective in treating AS, comprising the steps of:
(i) administering a test agent to a non-human animal model for AS prepared by the method of claim 30; and
(ii) determining whether at least one of AS condition/symptom is reduced or alleviated.
